(19) United States
(12) Patent Application Publication
(10) Pub. No.: US 2024/0115715 A1
(43) Pub. Date: Apr. 11, 2024

(54) PHARMACEUTICAL COMPOSITIONS FOR VAPORIZATION AND INHALATION

(71) Applicant: GROW BIOTECH PLC, London (GB)

(72) Inventors: Dominic Richard Pye, London (GB); Christopher James Cordier, London (GB); Sadaf Saad Anjum, London (GB); Benjamin Thomas Langley, London (GB); Ian Joseph Atkinson, London (GB)

(73) Assignee: GROW BIOTECH PLC, London (GB)

(21) Appl. No.: 18/541,510

(22) Filed: Dec. 15, 2023

Related U.S. Application Data
(63) Continuation of application No. PCT/GB2023/050420, filed on Feb. 23, 2023.
(60) Provisional application No. 63/268,467, filed on Feb. 24, 2022.

(51) Int. Cl. A61K 47/61; A61K 9/00; A61K 36/185; A61M 15/00

(52) U.S. Cl. CPC A61K 47/61; A61K 9/0073; A61K 36/185; A61M 15/003

(57) ABSTRACT

A pharmaceutical composition, a combination product including the composition and a method of manufacturing the composition. The composition includes a particulate complex of API bound with a crosslinked polysaccharide micro sponge. The combination product includes a dosage form of the particulate complex within a container. The amount of the particulate complex is selected to provide a defined dose of the API. The formulation may be heated within the container by a vaporizer device, vaporizing the API and dissociating the API from the micro sponge as vapour, which passes through apertures in the container facilitating administration of the API by inhalation of the vapour. The apertures are sized to facilitate passage of the vapour but prevent passage the particulate complex. The API may be hydrophobic such as phytocannabinoids or hydrophilic such as nicotine. The pharmaceutical composition may be manufactured by driving binding of the API to the micro sponge in solution.

### CROSS-REFERENCE TO RELATED APPLICATIONS

This patent application claims priority from U.S. Provisional Patent Application No. 63/268,467, filed Feb. 24, 2022 and entitled PHARMACEUTICAL COMPOSITIONS FOR VAPORIZATION AND INHALATION, the entirety of which is hereby incorporated by reference.

### FIELD

The present disclosure relates to pharmaceutical compositions, and combination products including the pharmaceutical compositions, for delivery of active pharmaceutical ingredients through vaporization and inhalation.

### BACKGROUND

Some pharmaceutical drugs include active pharmaceutical ingredients (“API”) that have slow absorption rates in gastric fluids, potentially decreasing bioavailability. Airway delivery may help address issues related to poor bioavailability, degradation, and adverse events in the gastrointestinal tract by avoiding first-pass metabolism in the liver. Airway delivery may provide easy access to large mucosal surfaces in the nose, paranasal sinuses, mouth, bronchus and lung, and thus may in some cases be well-suited for drug delivery. Inhalation may also be an attractive route for systemic drug delivery where rapid absorption and systemic effect are desired and injection or other invasive parenteral administration is preferably avoided.

There are several methods discussed in the literature for drug delivery through inhalation. Development and use of inhalation products originally focused on asthma, COPD and other conditions where pulmonary administration provides direct access to the site of action, and where a rapid onset of action is desirable. Pulmonary drug administration may facilitate safe and effective delivery where inhalation may be reproducibly applied to deliver a dose to a site of absorption in the respiratory tract. Inhalation may also facilitate providing an active dose while reducing the total dose to be given compared to oral administration, in part due to mitigating first pass liver metabolism.

### SUMMARY

In view of the shortcomings of previous approaches to pharmaceutical compositions suitable for vaporization and inhalation, there is motivation to provide a stable pharmaceutical composition that facilitates formulation, dosing and other handling, and that is suitable for vaporization and inhalation with low toxicity of the inhaled material.

Herein provided is a pharmaceutical composition including an active pharmaceutical ingredient (“API”) adsorbed onto, absorbed into, adhered with or otherwise non-covalently bound with, a crosslinked polysaccharide micro sponge to form a particulate complex. A micro sponge is a polymeric substance with a 3D microporous structure capable of absorbing molecules from a solution. A micro sponge as applied herein may include a porous polymeric substrate prepared from a crosslinked polysaccharide.

The pharmaceutical composition may be heated to release a thermal vapour of the API for inhalation. The crosslinked polysaccharide polymer micro sponge is used to capture the API by adsorption, absorption, adhering or other non-covalent binding of the API with the micro sponge, and the API is released as a vapour upon vaporization by application of heat with a vaporizer, without any carrier fluid in the composition. The crosslinked polysaccharide polymer micro sponge may be milled, ground or otherwise calibrated to a particular small particle size, then sieved for uniform particle size, so as to better define binding capacity of the cyclic polysaccharide monomeric units (such as cyclodextrin, α-cyclodextrin, β-cyclodextrin and γ-cyclodextrin) or other polysaccharides (such as maltodextrin, amylose and cellulose), and to increase the surface area of the micro sponge. A larger number of particles per gram of micro sponge increases the binding capacity as well as the rate of vaporization of the API.

The particulate complex may be provided as a component of a combination product that includes a sachet, dosage capsule, perforated tube or other container to facilitate dosage control as well as to make handling of the particulate complex simple. The combination product may be provided in blister packs or other multi-dosage form containers. The combination product may be heated by exposure of the container to elevated temperatures, causing the API within to vaporize from the particulate complex and pass through apertures in the container. The container may be shaped to easily interface with a specific vaporization device, or may be shaped and otherwise manufactured to facilitate compatibility with a range of common vaporization devices.

The API may include THC, CBD, other phytocannabinoids, other cannabis, terpenoids, flavonoids, alkaloids, heterogenous extracts of cannabis or other plants. The API may include DMT, 5-MeO-DMT, other tryptamines, nicotine, amphetamines, ephedrine, pseudoephedrine, menthol, or salts of any of the foregoing. Cannabis, nicotine or other APIs may be provided for purposes other than therapeutic purposes, and the pharmaceutical compositions formulated as the particulate complex, and the combination products including the particulate complex, may be prepared and used for non-therapeutic use, such as enjoyment of nicotine or cannabis.

The API may include a single purified molecule or a defined number of purified molecules. The API may include a heterogenous mixtures, such as a botanical extract, which may be a broad-spectrum plant extract. Mixtures of hydrophobic compounds from plant extracts may include a variety of hydrophobic compounds that bind to crosslinked polysaccharide polymer micro sponges. A single-molecule API may have a purity of 95% or greater. The API may be hydrophobic. The API may include THC, CBD, other phytocannabinoids, terpenoids, flavonoids, alkaloids, heterogenous extracts of cannabis or other plants. The API may be hydrophilic. The API may include DMT, 5-MeO-DMT, other tryptamines, nicotine, amphetamines, ephedrine, pseudoephedrine, other alkaloids, menthol, or salts of any of the foregoing.

The API itself may include terpenoids or other compounds that provide flavours or aromas to the API when vaporized, such as terpenoids and other compounds ordinarily present in a broad-spectrum botanical extract. Terpenoids or other flavouring agents may also be added to an API as an excipient, such as adding flavouring agents to nicotine, or phytocannabinoid distillate or isolate. The other ingredients, including flavouring agents such as terpenoids, or other excipients, may be bound with the micro sponge along with the API or separately from the API, such that the particulate complex may deliver different aromatic flavours when heated and the vapour of the API inhaled.

The crosslinked polysaccharide may be prepared to maximize surface to volume ratio, porosity, and available volume within a three-dimensional network of interconnected polysaccharides. The crosslinked polysaccharides may be formed of monomeric units of cyclic polysaccharides that each include a ring of six individual monosaccharide residues in α-cyclodextrin, seven individual monosaccharide residues in β-cyclodextrin or eight individual monosaccharide residues in γ-cyclodextrin. The crosslinked polysaccharides may be non-cyclic polysaccharides, in each case connected with one or more of a variety of crosslinkers.

Greater levels of crosslinking within the micro sponge may provide a 3D structure with a greater network of pores and a greater internal volume. The micro sponge may provide a loading capacity of the API to provide a particulate complex that includes a weight-by-weight ratio of the API to crosslinked polysaccharide of up to about 40%. In some embodiments, loading of the API may be between 1% and 40% of the mass of the crosslinked polysaccharide. In some embodiments, loading of the API may be between 5% and 25% of the mass of the crosslinked polysaccharide. In some embodiments, loading of the API may be between 10% and 20% of the mass of the crosslinked polysaccharide.

The crosslinked polysaccharide may include α-cyclodextrin, β-cyclodextrin, γ-cyclodextrin, other cyclodextrins, amylose, maltodextrin, cellulose, or modified versions of those crosslinked polysaccharides (e.g. alkylated, acetylated, carboxylated, aminated, etc.) or any suitable crosslinked polysaccharide polymer that provides a structure amenable to binding with the API.

General examples of suitable crosslinking agents include acyl halides, alkyl halides, pseudohalides, esters, isocyanides and acid anhydrides. Specific examples of suitable crosslinking agents include HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. Any ratio between 1:1 and 10:1 of crosslinking agent to cyclic polysaccharide monomer be applied for each cyclic polysaccharide monomer and each of the crosslinking agents.

The crosslinking agents may provide crosslinkers including hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl. Any ratio between 1:1 and 10:1 of crosslinker to cyclic polysaccharide monomeric unit may result for each crosslinked cyclic polysaccharide and each of the crosslinkers.

The crosslinked polysaccharide may include a β-cyclodextrin polymer. The crosslinked polysaccharide β-cyclodextrin polymer may be prepared with a ratio of 7:1 or 8:1 HMDI crosslinking agent to β-cyclodextrin monomers. The crosslinked polysaccharide β-cyclodextrin polymer may be prepared with a ratio of 7:1 or 8:1 ditosylate, sebacoyl chloride, adipoyl chloride, terephthaloyl chloride, other acyl chloride crosslinking agents to β-cyclodextrin monomers.

An API vaporization temperature at which the API vaporizes and dissociates from the crosslinked polysaccharide in the particulate complex is lower than a temperature at which combustion, melting, vaporization or other deformation of the crosslinked polysaccharide or the container occurs. The crosslinked polysaccharide used to prepare the micro sponge for the particulate complex is selected such that degradation, vaporization or combustion of the crosslinked polysaccharide is minimized or absent at a vaporization temperature of the API from the particulate complex. As a result, the vapour stream would have minimal or no degradation product or other byproducts of the crosslinked polysaccharide such as monomers or oligomeric units, which could be inhaled along with the vapour of the API.

The particulate complex may be manufactured into a variety of particle sizes using milling and sieving. Particulate complexes may have particle sizes of between 63 μm and 125 μm, 125 μm and 250 μm, 250 μm and 1,000 μm, or over 1,000 μm. Based on the properties of the crosslinked polymer, the particulate complexes are not expected to dissolve most solvents.

Portions of the particulate complex may be individually sealed and packaged in blister packs, sachets, perforated tubes or other perforated containers, dosage capsules, or other containers in order for dosage control as well as to make handling of the particulate complex simple. The complex and the container together may provide a combination product that includes both an API for consumption by a user of the combination product and a container within which the particulate complex may be heated, causing the API to vaporize for inhalation by a user. Sealing the dry powdered complex in a vapour permeable mesh or otherwise apertured container may strictly control the dosage as prescribed by the physician or otherwise intended for use as a dosage with a particular consumer packaged good. A combination product may include the particulate complex of the micro sponge bound with the API, which is within a container having perforations or other apertures in the container for allowing flow of vapour including the API through the perforations without particles of the micro sponge with bound API passing through the apertures.

Including the particulate complex within a vapour permeable container may facilitate vaporization of the API, accurate dosing and ease of handling the combination product. The container is prepared from a material that will not combust, melt, vaporize or otherwise degrade at or within a reasonable margin above the vaporization temperature of the API when the API is bound with the crosslinked polysaccharide in the particulate complex. The container may include a sachet with perforations or other apertures, a metal or ceramic cartridge or other suitable container for holding dosed amounts of the particulate complex as a dry powder.

Each composition including an API bound with a micro sponge may include a particular API with a unique vaporization temperature point. Workflow temperature settings of a vaporizer unit used with the composition may be predetermined based on the temperature necessary to release the API from the particulate complex and provide a thermal vapour for inhalation and administration of the API. Different compounds may require different vaporizer temperature settings that control the vaporization of the API.

The API may be released from the particulate complex by application of heat, vaporizing the API for administration to a subject by inhalation of vapour. The vapour includes vapour formed by heating of the particulate complex. Vaporization of the API and evolution of the API from the particulate complex as vapour occurs by energy introduction (e.g. heat, heat with vacuum, etc.). Evolution of the vapour from the pharmaceutical composition may be followed by cooling of the API prior to inhalation. Condensation upon cooling into vapour reaggregation into microdroplets is mitigated since the vapour phase occurs at low concentration in a laminar airflow. Cooling may take place in an airflow passage of a vaporizer between a heating chamber and a mouthpiece.

Upon application of heat to the particulate complex using a vaporization device, the API gains energy from the heat, overcomes the attraction between the API and the crosslinked polysaccharide, and the API vaporizes for inhalation. The larger the surface area of the crosslinked polysaccharide included in the pharmaceutical composition, the more easily the API may be vaporized and desorbed or otherwise dissociated from the crosslinked polysaccharide in the particulate complex. As a result, powders or other preparations, in which small sized particles are present, may provide advantages in terms of vaporization of API compared with a dried film of API on a planar solid substrate.

In addition to the API, the combination product may include and an additional payload. The additional payload may be an additional API or an excipient. The additional payload may, in combination with the API facilitate therapeutic benefits, such as alleviating specific conditions or ailments, or may provide particular flavours to cannabis products or nicotine products. The API and the additional payload may be bound with the same crosslinked polysaccharide, resulting in only one particulate complex. The API and the additional payload may be bound with separate crosslinked polysaccharides and mixed in the container, providing a mixed particulate complex. The API and the additional payload may be bound with separate crosslinked polysaccharides and sequestered in separate chambers within the container.

The API and the additional payload may each have distinct vaporization temperatures when complexed with the crosslinked polysaccharide. The API and the additional payload may be defined based on amounts of the API, the additional payload, or both, added proportional to the desired dosage of the API or the additional payload, or may be a result of the presence of different ratios of individual compounds in a heterogeneous API such as a botanical extract. The vapour may deliver more than one API, and possibly also flavourings or other ingredients, within the same stream of vapour. The nature of the vapour may also change as the composition is exposed to heat and individual components of the API, or the API and other ingredients, vaporize at different temperatures and rates, contributing different proportions to the vapour over time during vaporization.

The particulate complex used as a drug delivery system including the crosslinked polysaccharide polymer may provide advantages in precision of control of release kinetics, mixing uniformity, dose accuracy and dosing concentration as required for a given indication.

After washing and freeze drying, the pharmaceutical composition including the API bound with the micro sponge may be prepared for use in the form of a dried powder. A dried powder may be simpler to handle and work with than compositions of API that are resinous, sticky or otherwise difficult to work with. A dried powder also eliminates the need for carrier fluids, which are potentially harmful when vaporized incidentally to the API being vaporized.

Electronic vaporization systems such as e-cigarettes generally include a reservoir of liquid that is vaporized. The vaporization system may include nicotine or where permitted by law, phytocannabinoids. When a user inhales from an e-cigarette or similar device, a heater is activated to create vapourized API from a solution of the API in a liquid carrier fluid or from a heterogenous extract that includes the API. The API is then vaporized and may be inhaled by the user.

Serving only the purpose of a carrier fluid, reactions involving the common e-liquid solvents propylene glycol (“PG”) and vegetable glycerol or glycerin (“VG”) at corresponding vaping conditions have shown to result in evolution of toxic compounds in emissions (Talih et al. 2019; Son et al. 2020; Uchiyama et al. 2020). Elimination of the need for carrier fluid may mitigate risks associated with inhalation of toxic compounds resulting from degradation or vaporization of carrier fluid.

The particulate complexes provided herein may facilitate rapid vaporization, which may facilitate reducing the length of time during which the API is exposed to elevated temperatures for vaporization. Reducing the time that the API is exposed to elevated temperatures may mitigate thermal decomposition of the API, the crosslinked polysaccharide or both. The vaporized gas phase of the API rapidly enters the airflow of the vapour stream. Depending on the distance between the heat source heating up the API and a mouth piece, some of the vapour may coalesce into nanometer-sized aerosol particles during inhalation. Aerosol particles include a suspension of solid or liquid particles in a gas.

The prime purpose of the nasal and oral airway is to protect the lungs from hazardous exposures. Polymer release techniques have been investigated for the improvement of bioavailability and for physical stability to increase shelf life. Improvement of bioavailability and therapeutic efficacy has been previously attempted by maximizing alveolar absorption, while minimizing upper airway absorption. Small particles penetrate deep into the lungs depositing in the alveolar region (Heyder et al. 1986).

Factors related to the airway anatomy, physiology and aerodynamics that may severely limit the potential of delivery of API through inhalation have historically been challenging to address. By example, the powder complex must conform with applicable standards imposing limits on toxicity, irritation and immunogenicity. The crosslinked polysaccharide is large enough in particle size that the particulate complex, and the spent crosslinked polysaccharide are each sequestered within a mesh or other container with apertures to contain the crosslinked polysaccharide while vapour passes through the apertures. Both the particulate complex and the crosslinked polysaccharide would ideally be non-toxic and of low immunogenicity to mitigate the risk of harm in the event that the particulate complex or the crosslinked polysaccharide were to be inadvertently sloughed and inhaled by the user during vaporization. Similarly, if the vapour is not adequately cooled upon inhalation, there is a risk that the user's airway may be burned or irritated.

The pharmaceutical composition is a particulate complex that includes a micro sponge comprised of crosslinked polysaccharide polymer with a three-dimensional nanostructure suitable for binding with the API. In some cases where the crosslinked polysaccharide includes cyclic polysaccharide monomeric units, and without intending to be limited by any theory, the API may bind within the torus of cyclic polysaccharide monomeric units within the crosslinked polysaccharide polymer, and may also or alternatively be bound outside the torus of the crosslinked polysaccharide monomeric units within the crosslinked polysaccharide polymer. The API may be driven to adsorb with the crosslinked polysaccharide by dissolving the API in a solvent, adding the micro sponge to the solvent, then adding an antisolvent until the API preferentially binds to the crosslinked polysaccharide of the micro sponge rather than remaining in solution. Once the API is adsorbed with, absorbed by, adhered with or otherwise non-covalently bound with, the crosslinked polysaccharide of the micro sponge as a complex, the particulate complex is recovered from the solvent and antisolvent mixture. The complex may be recovered by physical separation where the micro sponge is insoluble. Where the micro sponge is also in solution, the particulate complex may be recovered by removal of the solvent and antisolvent mixture, leaving the particulate complex as a crystal, hydrate or precipitate.

The API may be bound with the crosslinked polysaccharide by solubilizing the API in a solvent and then adding an antisolvent in the presence of the crosslinked polysaccharide. The resulting complex may be separated from the antisolvent for drying and processing into the pharmaceutical compositions described herein. Other methods of binding with an API with the crosslinked polysaccharide micro sponge are possible. For example, the crosslinked polysaccharide micro sponge may be mixed with a solution of the API, and the solvent removed, similar to dry loading silica for flash column chromatography. Alternatively, the crosslinked polysaccharide micro sponge may be mixed with a solution of the API and physical or chemical means applied such that the solubility of the API in the solvent is reduced (for example change in pH, heating or cooling, change in the ionic strength of the solution, addition of an antisolvent).

The crosslinked polysaccharide micro sponge may be mixed with molten API and allowed to cool. The crosslinked polysaccharide micro sponge may be exposed to vapours of an API which penetrate the 3D structure of the crosslinked polysaccharide micro sponge and then condense on the micro sponge, binding with the crosslinked polysaccharide. The crosslinked polysaccharide may be soluble or insoluble in solvents for the API. The crosslinked polysaccharide may be soluble or insoluble in antisolvents for the API. A dry mixture of the particulate complex including the API bound with the crosslinked polysaccharide micro sponge may be subjected to mechanical force, such as grinding or milling. After grinding, milling or otherwise reducing the particle size, the resulting particulate complex may be sieved for a minimum particle size, or sequentially sieved for a uniform particle size. The complex of API and crosslinked polysaccharide may be recovered by physical separation where the micro sponge is insoluble. Where the micro sponge is also in solution, the particulate complex may be recovered by removal of the solvent and antisolvent mixture, leaving the particulate complex as a crystal, hydrate or precipitate.

In a first aspect, herein provided a pharmaceutical composition, a combination product including the composition and a method of manufacturing the composition. The composition includes a particulate complex of API bound with a crosslinked polysaccharide micro sponge. The combination product includes a dosage form of the particulate complex within a container. The amount of the particulate complex is selected to provide a defined dose of the API. The formulation may be heated within the container by a vaporizer device, vaporizing the API and dissociating the API from the micro sponge as vapour, which passes through apertures in the container facilitating administration of the API by inhalation of the vapour. The apertures are sized to facilitate passage of the vapour but prevent passage the particulate complex. The API may be hydrophobic such as phytocannabinoids or hydrophilic such as nicotine. The pharmaceutical composition may be manufactured by driving binding of the API to the micro sponge in solution.

In a further aspect, herein provided is a combination product comprising: a container comprising a container body, a chamber defined within the container body for receiving particulate material, and an aperture defined on the container body for providing fluid communication between the chamber and an external environment; and a particulate complex received within the chamber, the particulate complex comprising an API bound with a crosslinked polysaccharide, and the particulate complex having a minimum particle size. A largest dimension of the aperture is smaller than a smallest dimension of a particle having the minimum particle size for restricting flow of the particulate complex through the aperture and facilitating flow of vapour comprising the API from the chamber through the aperture. The crosslinked polysaccharide has a vaporization temperature, combustion temperature and melting temperature that are each higher than an API vaporization temperature at which the API vaporizes from the particulate complex.

In some embodiments, the container body comprises a rigid capsule. In some embodiments, the container body defines a mouth in fluid communication with the chamber for receiving the particulate complex, and the combination product further comprises a cap for sealing the mouth shut. In some embodiments, the container comprises a sachet. In some embodiments, the container body is formed from a container material selected from the group consisting of stainless steel, other metal, ceramic, woven mesh, silicone, plastics, other polymeric materials, filter paper fibres, hemp fibres and other fibres. In some embodiments, the aperture comprises a plurality of individual apertures, and each individual aperture has a largest dimension of the individual aperture that is smaller than the smallest dimension of a particle having the minimum particle size for restricting flow of the particulate complex through each individual aperture and facilitating flow of vapour comprising the API from the chamber through each individual aperture. In some embodiments, the minimum particle size is greater than 1,000 μm. In some embodiments, the minimum particle size is 250 μm to 1,000 μm. In some embodiments, the minimum particle size is 125 μm to 250 μm. In some embodiments, the minimum particle size is 63 μm to 125 μm.

In some embodiments, the particulate complex in the chamber provides a single dose of the API. In some embodiments, the API comprises a purified compound. In some embodiments, the API comprises a heterogenous mixture. In some embodiments, the heterogenous mixture comprises a botanical extract, a fungal extract or any other extract from biological material. In some embodiments, the heterogeneous mixture comprises a botanical extract from any plant within the genus Cannabis. In some embodiments, the API comprises a compound selected from the group consisting of a terpenoid, a flavonoid and an alkaloid. In some embodiments, the API comprises a compound selected from the group consisting of DMT, 5-MeO-DMT, other tryptamines, nicotine, an amphetamine, ephedrine, pseudoephedrine, other alkaloids, menthol or salts of any of the foregoing. In some embodiments, the API comprises a hydrophobic compound having an octanol:water partition coefficient of greater than 2. In some embodiments, the hydrophobic compound comprises a phytocannabinoid. In some embodiments, the phytocannabinoid comprises a phytocannabinoid selected from the group consisting of THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA. In some embodiments, the API comprises a hydrophilic compound having an octanol:water partition coefficient of 2 or lower.

In some embodiments, the crosslinked polysaccharide comprises a plurality of cyclodextrin monomeric units crosslinked by a cyclodextrin crosslinker; and the crosslinked polysaccharide was produced by reacting cyclodextrin monomers with a cyclodextrin crosslinking agent. In some embodiments, the cyclodextrin monomers are selected from the group consisting of α-cyclodextrin, β-cyclodextrin and γ-cyclodextrin. In some embodiments, the cyclodextrin crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, reacting the cyclodextrin monomers with the cyclodextrin crosslinking agent comprises reaction of a ratio of cyclodextrin crosslinking agent to cyclodextrin monomers; and the ratio of cyclodextrin crosslinking agent to cyclodextrin monomers is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the cyclodextrin crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl. In some embodiments, the crosslinked polysaccharide comprises a ratio of cyclodextrin crosslinker to cyclodextrin monomeric units; and the ratio of cyclodextrin crosslinker to cyclodextrin monomeric units is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the cyclodextrin crosslinker comprises hexamethylene dicarbamate; and the crosslinked polysaccharide is crosslinked with the cyclodextrin crosslinker at a ratio of 7:1 or 8:1 cyclodextrin crosslinker to cyclodextrin monomeric units. In some embodiments, the cyclodextrin crosslinker comprises a diester; and the crosslinked polysaccharide is crosslinked with the cyclodextrin crosslinker at a ratio of 7:1 or 8:1 cyclodextrin crosslinker to cyclodextrin monomeric units.

In some embodiments, the crosslinked polysaccharide comprises a non-cyclic polysaccharide crosslinked by a non-cyclic polysaccharide crosslinker; and the crosslinked polysaccharide was produced by reacting the non-cyclic polysaccharide with a non-cyclic crosslinking agent. In some embodiments, the non-cyclic polysaccharide is selected from the group consisting of amylose, maltodextrin and cellulose. In some embodiments, the non-cyclic crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, the non-cyclic crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl.

In some embodiments, the crosslinked polysaccharide comprises a modified crosslinked polysaccharide. In some embodiments, the modified polysaccharide comprises a modification selected from the group consisting of alkylation, acetylation, carboxylation and amination. In some embodiments, the API vaporization temperature is above 300° C. In some embodiments, the API vaporization temperature is between 300 and 250° C. In some embodiments, the API vaporization temperature is between 250° C. and 220° C. In some embodiments, the API vaporization temperature is between 220° C. and 200° C. In some embodiments, the API vaporization temperature is under 200° C. In some embodiments, the vaporization temperature, combustion temperature and melting temperature of the crosslinked polysaccharide are each at least 20° C. above the API vaporization temperature.

In some embodiments, the combination product includes an additional payload bound with the crosslinked polysaccharide. In some embodiments, the combination product an additional particulate complex received within the container, the additional particulate complex comprising an additional payload bound with an additional crosslinked polysaccharide, and the additional particulate complex having particles of an additional minimum particle size; and the additional crosslinked polysaccharide has an additional vaporization temperature, additional combustion temperature and additional melting temperature that are each higher than an additional payload vaporization temperature at which the additional payload vaporizes from the additional particulate complex. In some embodiments, the crosslinked polysaccharide and the additional crosslinked polysaccharide are crosslinked with different ratios of crosslinkers. In some embodiments, the crosslinked polysaccharide and the additional crosslinked polysaccharide are produced by crosslinking with different ratios of crosslinking agents. In some embodiments, a crosslinker that crosslinks the crosslinked polysaccharide is chemically distinct from an additional crosslinker that crosslinks the additional crosslinked polysaccharide. In some embodiments, a polysaccharide of the crosslinked polysaccharide is chemically identical to an additional polysaccharide of the additional polysaccharide. In some embodiments, a polysaccharide of the crosslinked polysaccharide is chemically distinct from an additional polysaccharide of the additional polysaccharide.

In some embodiments, the additional particulate complex is received within the chamber; and the largest dimension of the aperture is smaller than the smallest dimension of an additional particle having the additional minimum particle size for restricting flow of the additional particulate complex through the aperture and facilitating flow of vapour comprising the additional payload from the chamber through the aperture. In some embodiments, the combination product includes an additional chamber defined within the container body for receiving particulate material, and an additional aperture defined on the container body for providing fluid communication between the additional chamber and the external environment; wherein the chamber is sequestered from the additional chamber within the container body for preventing direct fluid communication between the chamber and additional chamber; the additional particulate complex is received within the additional chamber; and a largest dimension of the additional aperture is smaller than the smallest dimension of an additional particle having the additional minimum particle size for restricting flow of the additional particulate complex through the additional aperture and facilitating flow of vapour comprising the additional payload from the additional chamber through the additional aperture. In some embodiments, the additional aperture comprises a plurality of individual additional apertures, and each individual additional aperture has a largest dimension of the individual additional aperture that is smaller than the smallest dimension of an additional particle having the additional minimum particle size for restricting flow of the particulate complex through each individual additional aperture and facilitating flow of vapour comprising the additional payload from the additional chamber through each additional individual aperture. In some embodiments, contact between the API and the additional payload results in a reduced stability of the API or a reduced stability of the additional payload. In some embodiments, the reduced stability is a result of a chemical reaction selected from the group consisting of an acid/base reaction and condensation of a ketone with an amine.

In some embodiments, the additional payload comprises an additional API. In some embodiments, the additional API comprises an additional purified compound. In some embodiments, the additional API comprises an additional heterogenous mixture. In some embodiments, the additional heterogenous mixture comprises a botanical extract, a fungal extract or any other extract from biological material. In some embodiments, the additional heterogeneous mixture comprises a botanical extract from any plant within the genus Cannabis. In some embodiments, the additional API comprises an additional compound selected from the group consisting of a terpenoid, a flavonoid and an alkaloid. In some embodiments, the additional API comprises an additional compound selected from the group consisting of DMT, 5-MeO-DMT, other tryptamines, nicotine, an amphetamine, ephedrine, pseudoephedrine, other alkaloids, menthol or salts of any of the foregoing. In some embodiments, the additional API comprises an additional hydrophobic compound having an octanol:water partition coefficient of greater than 2. In some embodiments, the additional hydrophobic compound comprises a phytocannabinoid. In some embodiments, the phytocannabinoid comprises a phytocannabinoid selected from the group consisting of THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA. In some embodiments, the additional API comprises an additional hydrophilic compound having an octanol:water partition coefficient of 2 or lower.

In some embodiments, additional payload comprises an excipient. In some embodiments, the excipient comprises an excipient selected from the group consisting of an adjuvant, an antiadherant, a binder, a coating, a colour, a disintegrant, a glidant, a lubricant, a preservative and a sorbent. In some embodiments, the excipient comprises a compound for imparting a flavour or aroma when vaporized and inhaled, to provide the flavour or aroma to the vapour comprising the API. In some embodiments, the compound for imparting a flavour or aroma when vaporized and inhaled comprises a terpenoid. In some embodiments, the additional payload vaporizes at a temperature greater than the API vaporization temperature. In some embodiments, the additional payload vaporizes at a temperature equal to or below the API vaporization temperature.

In some embodiments, the additional minimum particle size is greater than 1,000 μm. In some embodiments, the additional minimum particle size is 250 μm to 1,000 μm. In some embodiments, the additional minimum particle size is 125 μm to 250 μm. In some embodiments, the additional minimum particle size is 63 μm to 125 μm. In some embodiments,

In some embodiments, the additional crosslinked polysaccharide comprises a plurality of additional cyclodextrin monomeric units crosslinked by an additional cyclodextrin crosslinker; and the additional crosslinked polysaccharide was produced by reacting additional cyclodextrin monomers with an additional cyclodextrin crosslinking agent. In some embodiments, the additional cyclodextrin monomers are selected from the group consisting of α-cyclodextrin, β-cyclodextrin and γ-cyclodextrin. In some embodiments, the additional cyclodextrin crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, reacting the additional cyclodextrin monomers with the additional cyclodextrin crosslinking agent comprises reaction of a ratio of additional cyclodextrin crosslinking agent to additional cyclodextrin monomers; and the ratio of additional cyclodextrin crosslinking agent to additional cyclodextrin monomers is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the additional cyclodextrin crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl. In some embodiments, the additional crosslinked polysaccharide comprises a ratio of additional cyclodextrin crosslinker to additional cyclodextrin monomeric units; and the ratio of additional cyclodextrin crosslinker to additional cyclodextrin monomeric units is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the additional cyclodextrin crosslinker comprises hexamethylene dicarbamate; and the additional crosslinked polysaccharide is crosslinked with the additional cyclodextrin crosslinker at a ratio of 7:1 or 8:1 additional cyclodextrin crosslinker to additional cyclodextrin monomeric units. In some embodiments, the additional cyclodextrin crosslinker comprises a diester; and the additional crosslinked polysaccharide is crosslinked with the additional cyclodextrin crosslinker at a ratio of 7:1 or 8:1 additional cyclodextrin crosslinker to additional cyclodextrin monomeric units.

In some embodiments, the additional crosslinked polysaccharide comprises an additional non-cyclic polysaccharide crosslinked by an additional non-cyclic polysaccharide crosslinker; and the additional crosslinked polysaccharide was produced by reacting the additional non-cyclic polysaccharide with an additional non-cyclic crosslinking agent. In some embodiments, the additional non-cyclic polysaccharide is selected from the group consisting of amylose, maltodextrin and cellulose. In some embodiments, the additional non-cyclic crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, the additional non-cyclic crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl.

In some embodiments, the additional crosslinked polysaccharide comprises a modified additional crosslinked polysaccharide. In some embodiments, the modified additional crosslinked polysaccharide comprises a modification selected from the group consisting of alkylation, acetylation, carboxylation and amination. In some embodiments, the additional payload vaporization temperature is above 300° C. In some embodiments, the additional payload vaporization temperature is between 300 and 250° C. In some embodiments, the additional payload vaporization temperature is between 250° C. and 220° C. In some embodiments, the additional payload vaporization temperature is between 220° C. and 200° C. In some embodiments, the additional payload vaporization temperature is under 200° C.

In some embodiments, the API comprises CBD; the crosslinked polysaccharide comprises a β-cyclodextrin cyclic polysaccharide crosslinked with hexamethylene dicarbamate crosslinkers; and the API vaporization temperature is 140° C.

In some embodiments, the API comprises CBG; the crosslinked polysaccharide comprises a β-cyclodextrin cyclic polysaccharide crosslinked with hexamethylene dicarbamate crosslinkers; and the API vaporization temperature is 220° C.

In some embodiments, the API comprises CBGA; the crosslinked polysaccharide comprises a β-cyclodextrin cyclic polysaccharide crosslinked with hexamethylene dicarbamate crosslinkers; and the API vaporization temperature is 220° C.

In some embodiments, the API comprises CBD; the crosslinked polysaccharide comprises a β-cyclodextrin cyclic polysaccharide crosslinked with sebacate crosslinkers; and the API vaporization temperature is 120° C.

In some embodiments, the API comprises CBD; the crosslinked polysaccharide comprises a β-cyclodextrin cyclic polysaccharide crosslinked with adipate crosslinkers; and the API vaporization temperature is 100° C.

In some embodiments, the API comprises CBD; the crosslinked polysaccharide comprises a β-cyclodextrin cyclic polysaccharide crosslinked with terephthalate crosslinkers; and the API vaporization temperature is 120° C.

In a further aspect, herein provided is a method of consuming an API by inhalation comprising providing the combination product as disclosed herein; heating the container to the API vaporization temperature, resulting in production of the vapour comprising the API; and inhaling the vapour comprising the API.

In some embodiments, the method includes cooling the vapour comprising the API prior to inhaling the vapour comprising the API. In some embodiments, the API vaporization temperature comprises a temperature at which at least 50% of the API is released from the particulate matter into the vapour comprising the API. In some embodiments, the API vaporization temperature comprises a temperature at which at least 75% of the API is released from the particulate matter into the vapour comprising the API. In some embodiments, the API vaporization temperature comprises a temperature at which at least 95% of the API is released from the particulate matter into the vapour comprising the API.

In a further aspect, herein provided is a method of manufacturing the combination product as described herein, including providing a solution comprising the API in a solvent; combining the crosslinked polysaccharide with the solution; combining an antisolvent with the solution, the API being less soluble in the antisolvent than in the solvent for facilitating binding of the crosslinked polysaccharide with the API, forming the particulate complex; isolating the particulate complex from the solution; drying the particulate complex; and transferring the particulate complex into chamber; wherein the crosslinked polysaccharide is insoluble in the antisolvent.

In some embodiments, the crosslinked polysaccharide is insoluble in the solvent. In some embodiments, the method includes milling or grinding the particulate complex to reduced the particle size of the particulate complex; and sieving the particulate complex after milling or grinding, for providing a minimum particle size of between 63 and 1,000 μm as disclosed herein.

In a further aspect, herein provided is a method of manufacturing the combination product as disclosed herein, the method comprising: providing a solution comprising the API and the additional payload in a solvent; combining the crosslinked polysaccharide with the solution; combining an antisolvent with the solution, the API and the additional payload each being less soluble in the antisolvent than in the solvent for facilitating binding of the crosslinked polysaccharide with the API and with the additional payload, forming the particulate complex; isolating the particulate complex from the solution; drying the particulate complex; and transferring the particulate complex into the container. The crosslinked polysaccharide is insoluble in the antisolvent.

In some embodiments, the crosslinked polysaccharide is insoluble in the solvent. In some embodiments, the method includes milling or grinding the particulate complex to reduced the particle size of the particulate complex; and sieving the particulate complex after milling or grinding, for providing a minimum particle size of between 63 and 1,000 μm as disclosed herein.

In a further aspect, herein provided is method of manufacturing a combination product as disclosed herein, the method comprising: providing a solution comprising the API in a solvent; combining the crosslinked polysaccharide with the solution; combining an antisolvent with the solution, the API being less soluble in the antisolvent than in the solvent for facilitating binding of the crosslinked polysaccharide with the API, forming the particulate complex; isolating the particulate complex from the solution; drying the particulate complex; transferring the particulate complex into the container; providing a solution comprising the additional payload in an additional solvent; combining the additional crosslinked polysaccharide with the additional solution; combining an additional antisolvent with the solution, the additional payload being less soluble in the additional antisolvent than in the additional solvent for facilitating binding of the additional crosslinked polysaccharide with the additional payload, forming the additional particulate complex; isolating the additional particulate complex from the solution; drying the additional particulate complex; and transferring the additional particulate complex into the container wherein the crosslinked polysaccharide is insoluble in the antisolvent; and the additional crosslinked polysaccharide is insoluble in the additional antisolvent.

In some embodiments, the crosslinked polysaccharide is insoluble in the solvent. In some embodiments, the additional crosslinked polysaccharide is insoluble in the additional solvent. In some embodiments, the method includes milling or grinding the particulate complex to reduced the particle size of the particulate complex; and sieving the particulate complex after milling or grinding, for providing a minimum particle size of between 63 and 1,000 μm as disclosed herein. In some embodiments, the method includes milling or grinding the additional particulate complex to reduced the additional particle size of the additional particulate complex; and sieving the additional particulate complex after milling or grinding, for providing a additional minimum particle size of between 63 and 1,000 μm as disclosed herein.

In a further aspect, herein provided is a pharmaceutical composition comprising an API bound with a crosslinked polysaccharide; wherein the particulate complex has a minimum particle size; the crosslinked polysaccharide has a vaporization temperature, combustion temperature and melting temperature that are each higher than an API vaporization temperature at which the API vaporizes from the particulate complex.

In some embodiments, the minimum particle size is greater than 1,000 μm. In some embodiments, the minimum particle size is 250 μm to 1,000 μm. In some embodiments, the minimum particle size is 125 μm to 250 μm. In some embodiments, the minimum particle size is 63 μm to 125 μm.

In some embodiments, the particulate complex in the chamber provides a single dose of the API. In some embodiments, the API comprises a purified compound. In some embodiments, the API comprises a heterogenous mixture. In some embodiments, the heterogenous mixture comprises a botanical extract, a fungal extract or any other extract from biological material. In some embodiments, the heterogeneous mixture comprises a botanical extract from any plant within the genus Cannabis. In some embodiments, the API comprises a compound selected from the group consisting of a terpenoid, a flavonoid and an alkaloid. In some embodiments, the API comprises a compound selected from the group consisting of DMT, 5-MeO-DMT, other tryptamines, nicotine, an amphetamine, ephedrine, pseudoephedrine, other alkaloids, menthol or salts of any of the foregoing. In some embodiments, the API comprises a hydrophobic compound having an octanol:water partition coefficient of greater than 2. In some embodiments, the hydrophobic compound comprises a phytocannabinoid. In some embodiments, the phytocannabinoid comprises a phytocannabinoid selected from the group consisting of THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA. In some embodiments, the API comprises a hydrophilic compound having an octanol:water partition coefficient of 2 or lower.

In some embodiments, the crosslinked polysaccharide comprises a plurality of cyclodextrin monomeric units crosslinked by a cyclodextrin crosslinker; and the crosslinked polysaccharide was produced by reacting cyclodextrin monomers with a cyclodextrin crosslinking agent. In some embodiments, the cyclodextrin monomers are selected from the group consisting of α-cyclodextrin, β-cyclodextrin and γ-cyclodextrin. In some embodiments, the cyclodextrin crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, reacting the cyclodextrin monomers with the cyclodextrin crosslinking agent comprises reaction of a ratio of cyclodextrin crosslinking agent to cyclodextrin monomers; and the ratio of cyclodextrin crosslinking agent to cyclodextrin monomers is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the cyclodextrin crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl. In some embodiments, the crosslinked polysaccharide comprises a ratio of cyclodextrin crosslinker to cyclodextrin monomeric units; and the ratio of cyclodextrin crosslinker to cyclodextrin monomeric units is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the cyclodextrin crosslinker comprises hexamethylene dicarbamate; and the crosslinked polysaccharide is crosslinked with the cyclodextrin crosslinker at a ratio of 7:1 or 8:1 cyclodextrin crosslinker to cyclodextrin monomeric units. In some embodiments, the cyclodextrin crosslinker comprises a diester; and the crosslinked polysaccharide is crosslinked with the cyclodextrin crosslinker at a ratio of 7:1 or 8:1 cyclodextrin crosslinker to cyclodextrin monomeric units.

In some embodiments, the crosslinked polysaccharide comprises a non-cyclic polysaccharide crosslinked by a non-cyclic polysaccharide crosslinker; and the crosslinked polysaccharide was produced by reacting the non-cyclic polysaccharide with a non-cyclic crosslinking agent. In some embodiments, the non-cyclic polysaccharide is selected from the group consisting of amylose, maltodextrin and cellulose. In some embodiments, the non-cyclic crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, the non-cyclic crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl.

In some embodiments, the crosslinked polysaccharide comprises a modified crosslinked polysaccharide. In some embodiments, the modified polysaccharide comprises a modification selected from the group consisting of alkylation, acetylation, carboxylation and amination. In some embodiments, the API vaporization temperature is above 300° C. In some embodiments, the API vaporization temperature is between 300 and 250° C. In some embodiments, the API vaporization temperature is between 250° C. and 220° C. In some embodiments, the API vaporization temperature is between 220° C. and 200° C. In some embodiments, the API vaporization temperature is under 200° C. In some embodiments, the vaporization temperature, combustion temperature and melting temperature of the crosslinked polysaccharide are each at least 20° C. above the API vaporization temperature.

In some embodiments, the additional payload comprises an additional API. In some embodiments, the additional API comprises an additional purified compound. In some embodiments, the additional API comprises an additional heterogenous mixture. In some embodiments, the additional heterogenous mixture comprises a botanical extract, a fungal extract or any other extract from biological material. In some embodiments, the additional heterogeneous mixture comprises a botanical extract from any plant within the genus Cannabis. In some embodiments, the additional API comprises an additional compound selected from the group consisting of a terpenoid, a flavonoid and an alkaloid. In some embodiments, the additional API comprises an additional compound selected from the group consisting of DMT, 5-MeO-DMT, other tryptamines, nicotine, an amphetamine, ephedrine, pseudoephedrine, other alkaloids, menthol or salts of any of the foregoing. In some embodiments, the additional API comprises an additional hydrophobic compound having an octanol:water partition coefficient of greater than 2. In some embodiments, the additional hydrophobic compound comprises a phytocannabinoid. In some embodiments, the phytocannabinoid comprises a phytocannabinoid selected from the group consisting of THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA. In some embodiments, the additional API comprises an additional hydrophilic compound having an octanol:water partition coefficient of 2 or lower.

In some embodiments, additional payload comprises an excipient. In some embodiments, the excipient comprises an excipient selected from the group consisting of an adjuvant, an antiadherant, a binder, a coating, a colour, a disintegrant, a glidant, a lubricant, a preservative and a sorbent. In some embodiments, the excipient comprises a compound for imparting a flavour or aroma when vaporized and inhaled, to provide the flavour or aroma to the vapour comprising the API. In some embodiments, the compound for imparting a flavour or aroma when vaporized and inhaled comprises a terpenoid. In some embodiments, the additional payload vaporizes at a temperature greater than the API vaporization temperature. In some embodiments, the additional payload vaporizes at a temperature equal to or below the API vaporization temperature.

In some embodiments, the additional minimum particle size is greater than 1,000 μm. In some embodiments, the additional minimum particle size is 250 μm to 1,000 μm. In some embodiments, the additional minimum particle size is 125 μm to 250 μm. In some embodiments, the additional minimum particle size is 63 μm to 125 μm. In some embodiments,

In some embodiments, the additional crosslinked polysaccharide comprises a plurality of additional cyclodextrin monomeric units crosslinked by an additional cyclodextrin crosslinker; and the additional crosslinked polysaccharide was produced by reacting additional cyclodextrin monomers with an additional cyclodextrin crosslinking agent. In some embodiments, the additional cyclodextrin monomers are selected from the group consisting of α-cyclodextrin, β-cyclodextrin and γ-cyclodextrin. In some embodiments, the additional cyclodextrin crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, reacting the additional cyclodextrin monomers with the additional cyclodextrin crosslinking agent comprises reaction of a ratio of additional cyclodextrin crosslinking agent to additional cyclodextrin monomers; and the ratio of additional cyclodextrin crosslinking agent to additional cyclodextrin monomers is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the additional cyclodextrin crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl. In some embodiments, the additional crosslinked polysaccharide comprises a ratio of additional cyclodextrin crosslinker to additional cyclodextrin monomeric units; and the ratio of additional cyclodextrin crosslinker to additional cyclodextrin monomeric units is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. In some embodiments, the additional cyclodextrin crosslinker comprises hexamethylene dicarbamate; and the additional crosslinked polysaccharide is crosslinked with the additional cyclodextrin crosslinker at a ratio of 7:1 or 8:1 additional cyclodextrin crosslinker to additional cyclodextrin monomeric units. In some embodiments, the additional cyclodextrin crosslinker comprises a diester; and the additional crosslinked polysaccharide is crosslinked with the additional cyclodextrin crosslinker at a ratio of 7:1 or 8:1 additional cyclodextrin crosslinker to additional cyclodextrin monomeric units.

In some embodiments, the additional crosslinked polysaccharide comprises an additional non-cyclic polysaccharide crosslinked by an additional non-cyclic polysaccharide crosslinker; and the additional crosslinked polysaccharide was produced by reacting the additional non-cyclic polysaccharide with an additional non-cyclic crosslinking agent. In some embodiments, the additional non-cyclic polysaccharide is selected from the group consisting of amylose, maltodextrin and cellulose. In some embodiments, the additional non-cyclic crosslinking agent is selected from the group consisting of HMDI, TMDI, isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. In some embodiments, the additional non-cyclic crosslinker is selected from the group consisting of hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl.

In some embodiments, the additional crosslinked polysaccharide comprises a modified additional crosslinked polysaccharide. In some embodiments, the modified additional crosslinked polysaccharide comprises a modification selected from the group consisting of alkylation, acetylation, carboxylation and amination. In some embodiments, the additional payload vaporization temperature is above 300° C. In some embodiments, the additional payload vaporization temperature is between 300 and 250° C. In some embodiments, the additional payload vaporization temperature is between 250° C. and 220° C. In some embodiments, the additional payload vaporization temperature is between 220° C. and 200° C. In some embodiments, the additional payload vaporization temperature is under 200° C.

In some embodiments, the API comprises CBD; the crosslinked polysaccharide comprises a β-cyclodextrin cyclic polysaccharide crosslinked with hexamethylene dicarbamate crosslinkers; and the API vaporization temperature is 140° C.

In some embodiments, the API comprises CBG; the crosslinked polysaccharide comprises a β-cyclodextrin cyclic polysaccharide crosslinked with hexamethylene dicarbamate crosslinkers; and the API vaporization temperature is 220° C.

In some embodiments, the API comprises CBGA; the crosslinked polysaccharide comprises a β-cyclodextrin cyclic polysaccharide crosslinked with hexamethylene dicarbamate crosslinkers; and the API vaporization temperature is 220° C.

In some embodiments, the API comprises CBD; the crosslinked polysaccharide comprises a β-cyclodextrin cyclic polysaccharide crosslinked with sebacate crosslinkers; and the API vaporization temperature is 120° C.

In some embodiments, the API comprises CBD; the crosslinked polysaccharide comprises a β-cyclodextrin cyclic polysaccharide crosslinked with adipate crosslinkers; and the API vaporization temperature is 100° C.

In some embodiments, the API comprises CBD; the crosslinked polysaccharide comprises a β-cyclodextrin cyclic polysaccharide crosslinked with terephthalate crosslinkers; and the API vaporization temperature is 120° C.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the attached Figures. In the attached Figures, features sharing a common final pair of numerals with a different first digit or digits correspond to equivalent features across embodiments shown in different figures (e.g. the container 62, the container 162, the container 262, the container 362, the container 462, the container 562, etc.).

FIG. 1 shows a combination product;

FIG. 2 shows a combination product with multiple individual apertures;

FIG. 3 shows a combination product with a sealed lid and an additional payload;

FIG. 4 shows a combination product with an additional chamber for an additional payload;

FIG. 5 shows a combination product with a sachet container;

FIG. 6 shows a combination product with a sachet container and an additional chamber for an additional payload;

FIG. 7 shows use of the combination product of FIG. 5 in a herbal vaporizer;

FIG. 8 shows a general schematic of a particulate complex including an API and an additional payload interacting with a crosslinked polysaccharide;

FIG. 9 shows a monomeric unit of a β-cyclodextrin polymer with one hexamethylene dicarbamate crosslinker in the monomeric unit;

FIG. 10 shows a crosslinked polymer of β-cyclodextrin monomeric units connected by hexamethylene dicarbamate crosslinkers;

FIG. 11 is a schematic diagram of an adsorption system;

FIG. 12 is a schematic diagram of the system of FIG. 11 with a crosslinked polysaccharide micro sponge added to the system;

FIG. 13 is a schematic diagram of the system of FIG. 11 with an API added to the system for binding with the crosslinked polysaccharide micro sponge;

FIG. 14 is a schematic diagram of the system of FIG. 11 with additional solvent added to the system;

FIG. 15 is a schematic diagram of the system of FIG. 11 with antisolvent added to the system;

FIG. 16 is a schematic diagram of the system of FIG. 11 while filtering a binding slurry to recover a target compounds;

FIG. 17 is a schematic diagram of the system of FIG. 11 while rinsing the filter with antisolvent;

FIG. 18 is a schematic diagram showing storage of a pharmaceutical composition provided by the system of FIG. 11;

FIG. 19 shows manufacture and use of a combination product including a pharmaceutical composition within a sachet;

FIG. 20 shows a plot of CBD vapourized from an hexamethylene dicarbamate-crosslinked β-cyclodextrin micro sponge, showing temperature on lefthand axis (dot dash line) and mass percent on righthand axis (dashed line);

FIG. 21 shows a plot of CBD vapourized from an hexamethylene dicarbamate-crosslinked β-cyclodextrin micro sponge, showing mass percent on lefthand axis (dashed line) and DSC (uV/mg) on righthand axis (solid line);

FIG. 22 shows a plot of CBD vapourized from an hexamethylene dicarbamate-crosslinked β-cyclodextrin micro sponge, showing temperature on lefthand axis (dot dash line) and DSC (uV/mg) on righthand axis (solid line);

FIG. 23 shows plots of CBD vapourized from crystalline CBD compared with CBD vapourized from an hexamethylene dicarbamate-crosslinked β-cyclodextrin micro sponge, with mass percent API from complex on lefthand axis (solid line), mass percent uncomplexed crystalline API (dashed line) on lefthand axis and temperature on righthand axis (dot-dash line);

FIG. 24 shows a plot of CBG vapourized from an hexamethylene dicarbamate-crosslinked β-cyclodextrin micro sponge, showing temperature on lefthand axis (dot dash line) and mass percent on righthand axis (dashed line);

FIG. 25 shows a plot of CBG vapourized from an hexamethylene dicarbamate-crosslinked β-cyclodextrin micro sponge, showing mass percent on lefthand axis (dashed line) and DSC (uV/mg) on righthand axis (solid line);

FIG. 26 shows a plot of CBG vapourized from an hexamethylene dicarbamate-crosslinked β-cyclodextrin micro sponge, showing temperature on lefthand axis (dot dash line) and DSC (uV/mg) on righthand axis (solid line);

FIG. 27 shows plots of CBG vapourized from crystalline CBG compared with CBG vapourized from an hexamethylene dicarbamate-crosslinked β-cyclodextrin micro sponge, with mass percent API from complex on lefthand axis (solid line), mass percent uncomplexed crystalline API (dashed line) on lefthand axis and temperature on righthand axis (dot-dash line);

FIG. 28 shows a plot of CBGA vapourized from an hexamethylene dicarbamate-crosslinked β-cyclodextrin micro sponge, showing temperature on lefthand axis (dot dash line) and mass percent on righthand axis (dashed line);

FIG. 29 shows a plot of CBGA vapourized from an hexamethylene dicarbamate-crosslinked β-cyclodextrin micro sponge, showing mass percent on lefthand axis (dashed line) and DSC (uV/mg) on righthand axis (solid line);

FIG. 30 shows a plot of CBGA vapourized from an hexamethylene dicarbamate-crosslinked β-cyclodextrin micro sponge, showing temperature on lefthand axis (dot dash line) and DSC (uV/mg) on righthand axis (solid line);

FIG. 31 shows plots of CBGA vapourized from crystalline CBGA compared with CBGA vapourized from an hexamethylene dicarbamate-crosslinked β-cyclodextrin micro sponge, with mass percent API from complex on lefthand axis (solid line), mass percent uncomplexed crystalline API (dashed line) on lefthand axis and temperature on righthand axis (dot-dash line);

FIG. 32 shows a plot of CBD vapourized from a sebacoyl-crosslinked β-cyclodextrin micro sponge, showing temperature on lefthand axis (dot dash line) and mass percent on righthand axis (dashed line);

FIG. 33 shows a plot of CBD vapourized from a sebacoyl-crosslinked β-cyclodextrin micro sponge, showing mass percent on lefthand axis (dashed line) and DSC (uV/mg) on righthand axis (solid line);

FIG. 34 shows a plot of CBD vapourized from a sebacoyl-crosslinked β-cyclodextrin micro sponge, showing temperature on lefthand axis (dot dash line) and DSC (uV/mg) on righthand axis (solid line);

FIG. 35 shows a plot of CBD vapourized from an adipoyl-crosslinked β-cyclodextrin micro sponge, showing temperature on lefthand axis (dot dash line) and mass percent on righthand axis (dashed line);

FIG. 36 shows a plot of CBD vapourized from an adipoyl-crosslinked β-cyclodextrin micro sponge, showing mass percent on lefthand axis (dashed line) and DSC (uV/mg) on righthand axis (solid line);

FIG. 37 shows a plot of CBD vapourized from an adipoyl-crosslinked β-cyclodextrin micro sponge, showing temperature on lefthand axis (dot dash line) and DSC (uV/mg) on righthand axis (solid line);

FIG. 38 shows a plot of CBD vapourized from a terephthaloyl-crosslinked β-cyclodextrin micro sponge, showing temperature on lefthand axis (dot dash line) and mass percent on righthand axis (dashed line);

FIG. 39 shows a plot of CBD vapourized from a terephthaloyl-crosslinked β-cyclodextrin micro sponge, showing mass percent on lefthand axis (dashed line) and DSC (uV/mg) on righthand axis (solid line); and

FIG. 40 shows a plot of CBD vapourized from a terephthaloyl-crosslinked β-cyclodextrin micro sponge, showing temperature on lefthand axis (dot dash line) and DSC (uV/mg) on righthand axis (solid line).

### DETAILED DESCRIPTION

Generally, the present disclosure provides pharmaceutical compositions and formulations suitable for delivery of an API through inhalation, a combination product including a container holding the compositions and formulations, and methods for preparing the pharmaceutical composition. The pharmaceutical composition includes an API bound with a crosslinked polysaccharide to form a particulate complex. The complex may be formulated and otherwise prepared as a dried powder, facilitating handling of the particulate complex. The crosslinked polysaccharide provides a micro sponge, which is a polymeric substance having a microporous structure capable of binding with molecules from solution or otherwise. The API is bound with the crosslinked polysaccharide micro sponge by absorption, adsorption, adhering or other non-covalent binding. Upon heating of the particulate complex, the API vaporizes and may be inhaled, leaving the crosslinked polysaccharide behind. The container includes apertures sized to allow vapour including the API to flow out of the container, while retaining spent crosslinked polysaccharide micro sponge particulate within the container.

A combination product is product that includes an API and a device to facilitate delivery and use of the API. When the API is provided for a therapeutic purpose, the combination product may include a medical device and a drug product or natural health product, with the drug product or natural health product including the API, and the medical device facilitating delivery or use of the drug product or natural health product. In these cases, combination products are sometimes referred to as drug-device combinations or drug-modified medical devices. When the API is provided for a non-therapeutic purpose, such as cannabis use, the combination product may include a cannabis accessory and cannabis, with the cannabis including the API and the cannabis accessory facilitating delivery or use of the cannabis. When the API is provided for a non-therapeutic purpose, such as nicotine use, the combination product may include a vaping accessory and nicotine, with API including nicotine and the vaping accessory facilitating delivery or use of the nicotine. The scope of the pharmaceutical composition, and the combination device including the pharmaceutical composition, shall not be limited by a particular regulatory category, or by the presence or absence of a therapeutic benefit in relation to a particular application of the pharmaceutical composition, and the combination device including the pharmaceutical composition.

The particulate complex may be suitable for use in delivering the API through vaporization and inhalation of the API with mitigated or absent combustion, vaporization or degradation of the crosslinked polysaccharide micro sponge substrate. When the particulate complex is heated, a temperature of the particulate complex may be achieved that exceeds an API vaporization temperature at which the API vaporizes from the particulate complex, and the API may be inhaled by a user. Inhalation of vapour into the user's airway delivers the API into a site for absorption of the API by the user. The crosslinked polysaccharide remains in solid particulate form and does not enter the vapour phase at the API vaporization temperature. The API may be heated in a herbal vaporizer, a medical device, a liquid vaporizer, or similar device by passing current through a heating element, which in turn heats the air around the particulate complex by convection heating, heats the particulate complex itself through conduction heating, or otherwise heats the particulate complex to vaporize the API.

The API vaporization temperature is below temperatures at which the API, the crosslinked polysaccharide or the container combusts, or at which the crosslinked polysaccharide or the container vaporizes, melts or otherwise deforms. As a result, neither the crosslinked polysaccharide, nor any crosslinked polysaccharide degradation byproducts, nor any container degradation products are inhaled or consumed along with the API. The API vaporization temperature may be at least 20° C. below temperatures at which the API, the crosslinked polysaccharide or the container combusts, or at which the crosslinked polysaccharide or the container vaporizes, melts or otherwise deforms. The API vaporization temperature may have a margin of 20° C., 25° C., 30° C., 35° C., 40° C., 45° C., 50° C., 55° C., 60° C., 65° C., 70° C., 75° C., 80° C., 85° C., 90° C., 95° C., or 100° C. below temperatures at which the API, the crosslinked polysaccharide or the container combusts, or at which the crosslinked polysaccharide or the container vaporizes, melts or otherwise deforms. At the temperatures used to elute the API from the micro sponge as a vapour, the API is in a vapour phase to facilitate inhalation of the API. The API may be cooled prior to inhalation where the API vaporization temperature is elevated to an extent that cooling prior to inhalation may provide an improved user experience.

Soluble cyclodextrins have been used to bind fragrances, which can be released upon heating. Such reagents are capable of releasing fragrances during ironing or when heated by the human body. These approaches may be applied to a dryer sheet where the heat from a clothes dryer releases the fragrance into the clothing. Such applications, in contrast with the current pharmaceutical compositions, may include application of hydrophilic soluble complexes between a fragrance and a polysaccharide, where the polysaccharide is not crosslinked. In contrast, crosslinked polysaccharides, particularly crosslinked polysaccharides with greater degrees of crosslinking, may be insoluble in many solvents. The crosslinked polysaccharide may be insoluble in solvents that solubilize the API. The crosslinked polysaccharide may be insoluble in antisolvents that do not solubilize the API.

The particulate complex between the API and the crosslinked polysaccharide may be milled, ground or otherwise prepared at a defined particle size. As used herein, the particle size is the diameter of spherical particles, or the smallest dimension of a non-spherical particle. The particle size may be matched with a container such that apertures in the container are sized appropriately to retain the particulate complex and also to retain the spent crosslinked polysaccharide after vaporization of the API and evolution of the API from the particulate complex. The diameter for circular apertures, or the largest dimension for non-circular apertures, of each aperture is smaller than the smallest dimension of a particle having the particle size of the particulate complex, and of a particle having the particle size of the spent crosslinked polysaccharide, for preventing passage of the particulate complex or of the crosslinked polysaccharide through the aperture.

Inhalation of an API as vapour released from the crosslinked polysaccharide micro sponge may be effected by using a vaporization device. Vaporization of the API is from the dry powder particulate complex included within a rigid capsule, sachet, or other container as a combination product. The combination product mitigates the need for carrier fluid, solvent or “e-juice” such as PG or VG. A temperature setting may be selected on the vaporizer that best suits release of the API. The vaporizer may for example include a Volcano Medic hybrid. For phytocannabinoid API, the vaporizer may be set to a setting of between 180° C. and 230° C., which includes the vaporization temperatures for common phytocannabinoids. The dosage capsule, mesh sachet, perforated metal or ceramic container, or similar vapour-porous container retains the polymer into the vaping device.

The container includes a defined weight and dosage of the particulate complex selected to provide a defined dosage of the API. The vaporizer may be pre-heated prior to a consumption event starting or may be heated during consumption of the API. The complex is heated to vaporize the API, resulting in vapourized API. The API vapour may be consumed directly from the device as the vapour evolves from the particulate complex between the API and the crosslinked polysaccharide. The vapour may be channeled into a bag to allow the vapour to cool before consumption, the user may then consume the API from the bag by inhaling the dose-specific vapour. The combination product facilitates use of the API in a clinical trial with a uniformly dosed and controlled administration of the API based on easily the quantifiable particulate complex. Where the particulate complex is between about 10% and 20% API by weight, then hundreds of milligrams of the particulate complex may be portioned out to provide tens of milligrams of the API during a consumption event.

Dose control of a single compound inhalation can be based on a known weight of complex at a known concentration added to the vaporizer. When heated to release a known amount of vapour, dose control of the API may be calibrated to a particular amount of the API per inhalation consumption event of a defined duration. Under controlled conditions as a means of thermal release, use of the particulate complex as a formulation facilitates specific dose control of the inhaled therapy of one or more API isolates complexed with the crosslinked polysaccharide.

The pharmaceutical compositions provided herein facilitate rapid systemic delivery of API through inhalation by volatilization from solid particulate complexes between the API and a crosslinked polysaccharide, without use of carrier fluids. Use of a solid-form complex with API bound with a crosslinked polysaccharide may mitigate risks related to contamination, oxidative losses, and potential toxicity from a carrier fluid that carries no therapeutic benefit. The vapour may be delivered through a dose inhaler or vaporization device.

A method for the adsorption of hydrophobic compounds to a cyclic polysaccharide for purification of the hydrophobic compounds is described in WO 2021/090003. Briefly, a mixture of a hydrophobic compound in a hydrophobic solvent is exposed to an insoluble cyclic polysaccharide. Water or saline solution is added to the hydrophobic solvent to provide a driving force for the hydrophobic compound to bind with the insoluble cyclic polysaccharide. The insoluble cyclic polysaccharide with bound hydrophobic compound is then exposed to a lipophilic solvent to recover the hydrophobic compound. The principles of solvent and antisolvent may be applied to drive binding of the API to the crosslinked polysaccharide in the present case.

Binding of the API with the crosslinked polysaccharide to provide the particulate complex between the API and polymers of a crosslinked polysaccharide for heating to vaporize and release the API as a thermal vapour for inhalation, while leaving the crosslinked polysaccharide behind, may provide advantages in terms of formulation and handling of the composition, relative to handling of API that is not complexed with the crosslinked polysaccharide. Where the API is a hydrophobic API that is ordinarily resinous, viscous and difficult to physically work with, preparing a complex with the crosslinked polysaccharide may facilitate providing a dried powder or other complex that is simple to formulate, flow, sift, store, transport, handle, vaporize and otherwise work with, compared with viscous APIs or APIs solubilized in carrier fluids.

Drying equipment may be used to remove the remainder of any fluid from the particulate complex powder, without removing significant amounts of the API. With no need for a carrier fluid in the composition, no risk or perceived risk from polyhydric alcohols (e.g. from PG, VG, etc.) would be associated with the composition provided herein. Carrier fluid vaporization may result when vaporizing API that is dissolved in a carrier fluid. Carrier fluid vaporization may result in an aerosol by vaporizing of a consumable e-liquid, such as a liquid composition prepared from PG, VG, water, nicotine and flavours. The liquid composition including the carrier fluid may be drawn by a wicking material into a resistive heating coil in which it is heated and evaporated. Carrier fluids carry the risk of consumption or carrier fluid aerosols, leaking of the liquid composition though gaskets and the mouthpiece, inefficient heating and an inconsistent aerosol composition.

Binding of the API with the crosslinked polysaccharide micro sponge and maintenance of the API as part of the particulate complex may protect the API from oxygen and moisture, increasing stability relative to the API that is not bound with the crosslinked polysaccharide micro sponge. Since the API is molecularly dispersed throughout the matrix of the crosslinked polysaccharide micro sponge, rather than forming areas of bulk API, reaction of the API with itself may be prevented, and autocatalytic degradation processes may be retarded. In addition, polymorphism of the API is mitigated by formulating an API in a complex with the micro sponge. Without intending to be limited by any theory, the molecularly dispersed nature of the API is suggested by the lack of a melting point identified for the API in TGA analyses of the particulate complex.

As the API is to be administered directly to the lung as a vapour, dissolution of the bulk API in aqueous solutions such as stomach or intestinal fluids is not required, mitigating need to define dissolution characteristics of different polymorphs or other solid forms of the API. Inhalation of API may be less sensitive to first pass liver metabolism than oral administration. Inhalation of API may provide faster onset time for the API compared with oral administration.

A controlled dry complex may facilitate limiting the number of individual compounds from a botanical extract or other heterogenous API, such as a broad-spectrum cannabis extract, that are bound with the micro sponge to a smaller subset. By removing water-soluble extract components, vaporization may be limited to a defined subset of hydrophobic compounds. By removing lipid-soluble extract components, vaporization may be limited to a defined subset of hydrophilic compounds. Control may be applied during formulation to limit the presence, and therefore the inhalation, of compounds that may negatively impact safety, and that do not contribute to therapeutic benefits or to subjectively important effects (such as with adult use cannabis products). Use of the particulate complex may facilitate maintaining control over batch-to-batch reproducibility during manufacture of the particulate complex.

Complexation between an API and a crosslinked polysaccharide may be used to improve the chemical, physical and thermal stability of the API. For an active molecule to degrade upon exposure to oxygen, water, radiation or heat, chemical reactions must take place. Where an API is bound with the crosslinked polysaccharide, the API may be less available for reactants to destabilize the API from the particulate complex or otherwise react with the API. API bound with a micro sponge may have a greater stability than API otherwise formulated. Non-covalent particulate complexes may provide improved physicochemical characteristics when compared with the API alone, including better stability during storage and shipment, increased bioavailability, fewer undesirable side effects, higher vaporization temperature, and tighter dosage control when generating a vapour.

Complexation of an API with a crosslinked polysaccharide may change the vaporization temperature of the API. In some cases, thermal release of the API from the particulate complex may occur at temperatures lower, equal to or higher than the vaporization temperature of the API in the absence of the crosslinked polysaccharide.

API and Excipients

The API may be provided for therapeutic purposes in which delivery of the API to alveoli provides a safe and effective route of administration for a particular therapeutic indication. The API may also be provided for purposes other than therapeutic purposes. In non-therapeutic applications, the pharmaceutical formulation of the particulate complex, and inclusion of the particulate complex in a container, may be used for reasons such as enjoyment of nicotine, cannabis or flavoured vapour.

The API may include THC, CBD, other phytocannabinoids, terpenoids, flavonoids, alkaloids, heterogenous extracts of cannabis or other plants, DMT, 5-MeO-DMT, other tryptamines, nicotine, amphetamines, ephedrine, pseudoephedrine, menthol, salts of any of the foregoing, or any suitable API. The API may have a vaporization temperature of under 300° C., under 250° C., under 220° C., under 200° C. or other suitable vaporization temperatures.

The API may include an isolated phytocannabinoid, an isolated flavonoid, an isolated alkaloid, an isolated tryptamine, nicotine, an isolated amphetamine, or any other isolated and purified compound. The API may include a defined mixture of phytocannabinoids, a defined mixture of phytocannabinoids and terpenoids, a defined mixture of terpenoids, a defined mixture of flavonoids, a defined mixture of alkaloids, a defined mixture of tryptamines, nicotine mixed with flavour or other compounds, a defined mixture of amphetamines, ephedrine with other compounds, pseudoephedrine with other compounds, menthol with other compounds, a heterogenous cannabis extract or other mixtures that may provide a complex useful as a pharmaceutical composition and that result in a suitable API vaporization temperature from the particulate complex.

The API may include one or more hydrophobic compounds, such as phytocannabinoids, isopropanoids, terpenoids or other compounds extracted from cannabis, phytocannabinoids synthesized through biosynthesis or chemically, phytocannabinoid analogues, or other similar compounds, and may include an extract or other heterogenous API that includes more than one specific hydrophobic compound. The API may include a heterogenous extract enriched with one or more particular phytocannabinoids, isopropanoids, terpenoids or other bioactive hydrophobic compounds following extraction of heterogeneous extracts from plant biomass.

Mixed chemical compositions may be bound with the crosslinked polysaccharide micro sponges to provide the particulate complex. Broad-spectrum heterogenous plant biomass extracts may be prepared and the hydrophobic components that can bind to the crosslinked polysaccharide may be captured and bound with the crosslinked polysaccharide. Compared with a heterogenous botanical extract, heat elution of a single-molecule API, or small number of single molecules as API, and may result in a more consistent, and better-characterized output, and an output having greater purity, compared with the starting extract solution.

Phytocannabinoids are a diverse group of chemical compounds and may include delta-9-tetrahydrocannabinol (“THC”), delta-9-tetrahydrocannabinolic acid (“THCA”), cannabidiol (“CBD”), cannabidiolic acid (“CBDA”), cannabinol (“CBN”), cannabigerol (“CBG”), cannabigerolic acid (“CBGA”), cannabichromene (“CBC”), cannabichromenic acid (“CBCA”), cannabielsoin (“CBE”), cannabielsoinic acid (“CBEA”), cannabicyclol (“CBL”), cannabicyclolic acid (“CBLA”), iso-tetrahydrocannabinol (“iso-THC”), iso-tetrahydrocannabinolic acid (“iso-THCA”), cannabicitran (“CBT”), cannabicitrannic acid (“CBTA”), delta-8-tetrahydrocannabinol (“Δ8THC”), delta-8-tetrahydrocannabinolic acid (“Δ8THCA”), delta-9-tetrahydrocannabivarin (“THCV”), delta-9-tetrahydrocannabivarinic acid (“THCVA”), cannabidivarin (“CBDV”), cannabidivarinic acid (“CBDVA”). The API may include THC, CBD, other phytocannabinoids, terpenoids, flavonoids, heterogenous extracts of cannabis or other plants, or other hydrophobic APIs.

The API may include compounds such as nicotine, N,N-dimethyltryptamine (“DMT”), 5-methoxy-N,N-dimethyltryptamine (“5-MeO-DMT”), other tryptamines, amphetamines, ephedrine, pseudoephedrine, other alkaloids, menthol, or salts of any of the foregoing. Theses classes APIs include both hydrophobic APIs and hydrophilicAPIs. Hydrophobic compounds included as APIs may have an octanol:water partition coefficient of greater than 2. Hydrophilic compounds included as APIs may have an octanol:water partition coefficient of 2 or lower.

APIs are commonly formulated as salts, which may improve stability and dissolution rate, and control polymorphism (Wiedmann et al., 2016). When formulating an API for administration by vaporisation, forming a salt may increase the vaporisation temperature of the API, in some cases to the point where the API may be more likely to decompose or combust rather than vaporise. As a result, for vaporization, some APIs may be preferably provided as a freebase or free acid. Use of the crosslinked polysaccharide micro sponge in formulating an API for administration by vaporisation may provide advantages in terms of stability, in terms of mitigating variability due to polymorphism and in terms of ease of handling. For some compounds, these advantages that may otherwise be provided by preparing a salt of the API, and formulation of the API into the particulate complex may provide these advantages without creating salts for some APIs, mitigating motivation for preparing a salt of the API.

A single API may include multiple compounds, and in some cases an unknown total number of compounds, particularly outside of therapeutic contexts, such as broad spectrum cannabis extracts. Two or more APIs may be provided to a user in the same inhalation event. Distinct APIs may be bound separately to provide unique polymer-API complex compositions. After each complex composition is quantified, known amounts of each different API-micro sponge complex may be separately measured and added for combination into a single dose with two or more APIs during manufacturing of the combination product. Adding a formulation that includes two different particulate complexes to a vapour-permeable container facilitates simultaneous handling and simultaneous vapour release of multiple APIs for inhalation upon application of heat to the particulate complex.

Pharmaceutically acceptable excipients may be bound with the crosslinked polysaccharide polymer micro sponge along with the API. Pharmaceutically acceptable excipients may or may not be volatilised and inhaled by the user along with the API when heated. Pharmaceutically acceptable excipients may include adjuvants, antiadherants, binders, coatings, colours, disintegrants, flavours, glidants, lubricants, preservatives and sorbents. It may be desirable that the pharmaceutically acceptable excipient is volatilised and inhaled by the user along with the API, such as in the case of sweeteners or other flavourants. It may also be that, while not a desired feature that the pharmaceutically acceptable excipient is volatilised and inhaled by the user along with the API, it is not detrimental to the user's health or vaping experience, such as where the pharmaceutically acceptable excipient has no flavour and minimal toxicity profile when inhaled into the lungs.

Excipients included along with API in the combination product may include flavouring or flavour-masking components that bind to the same crosslinked polysaccharide as the API. Excipients included along with the API in the combination product may include flavouring or flavour-masking components bound to an additional crosslinked polysaccharide, while the API is bound with the crosslinked polysaccharide. The crosslinked polysaccharide and the additional crosslinked polysaccharide may be the same type of crosslinked polysaccharide or different crosslinked polysaccharide.

Excipients that are flavouring, flavour-masking compounds or other flavourants may be used to create a desired taste or aroma in a vapour product for adult consumers (e.g. licorice, hydrangea, Japanese white bark magnolia leaf, chamomile, fenugreek, clove, menthol, Japanese mint, aniseed, cinnamon, herb, wintergreen, cherry, berry, peach, apple, flavour enhancers, bitterness receptor site blockers, sensorial receptor site activators or stimulators, sugars, sucralose, acesulfame potassium, aspartame, saccharine, cyclamates, lactose, sucrose, glucose, fructose, sorbitol, mannitol or other sugar substitutes, charcoal, chlorophyll, minerals, botanicals, breath freshening agents, etc.). Flavouring or flavour-masking agents may be imitation, synthetic or natural ingredients or blends thereof.

Particle Size

The particulate complex is made up of particles of the crosslinked polysaccharide with the API bound to the crosslinked polysaccharide. The particle diameter or other aspects of the particle size may be selected from a range of reasonable particle sizes. The particle size of the crosslinked polysaccharide making up the micro sponge may be selected based on a particular application. Greater particle size facilitates handling and allows for larger apertures in the container. Smaller particle size facilitates binding of the API with the crosslinked polysaccharide and vaporization of the API.

The particle size is sufficiently large to ensure that the aperture of the container retains the particulate complex within the chamber, and where applicable retain the additional particulate complex within the additional chamber. The particle sizes being larger than the apertures mitigates loss of particulate complex prior to heating and possible inhalation of particulate complex or of spent crosslinked polysaccharide micro sponge. The particle size may be selected to be sufficiently small to increase binding capacity for the API per gram of the crosslinked polysaccharide micro sponge. A smaller polymeric microparticle size may facilitate potential based on surface area for capturing the API, complexing and binding with the API, and releasing the API upon vaporization. When ground, milled or otherwise reduced to a smaller diameter, the surface area per gram of micro sponge increases, and the capture capacity of API per gram of crosslinked polysaccharide micro sponge is increased relative to more coarse particles of the crosslinked polysaccharide micro sponge.

Particle sizes may include high surface area small particles, such as particle sizes between 63 to 125 μm, or such as particle sizes between 125 to 250 μm, which may increase efficiency of or otherwise facilitate binding between the API and the crosslinked polysaccharide, and may facilitate vaporization of the API from the particulate complex. Moderate particle size (250 to 1,000 μm) may facilitate for ease of handling of the particulate complex and for use of apertures in the container that exclude these larger particle sizes. Large particles size (>1,000 μm) may be applied for large scale applications and to facilitate use of larger apertures in the container.

Combination Product

Dosage forms of the powdered particulate complex may be prepared by including the particulate complex as a payload within a combination product that also includes a capsule, a mesh sachet or other vapour-permeable container for easy handling, heating and inhalation of a pre-quantified dose of the API. A capsule may include a rigid body that is crimped or capped shut. A sachet is a flexible bag and may include woven mesh screens providing apertures through which vapour including the API may flow during administration to a user.

The sachet includes mesh, perforations, pores or other apertures sized small enough to hold the particulate complex, and the spent crosslinked polysaccharide, within the container during inhalation and prevent escape during inhalation. An aperture size smaller than the particle size of the particulate complex, and also smaller than the particle size of the exhausted crosslinked polysaccharide, sequesters the micro sponge within the sachet, both while bound with API in the particulate complex and once exhausted of API after heating. The apertures are also sized to be sufficiently large to facilitate uninterrupted and smooth flow of the vapour of the API. The sachet may be manufactured from a container material such as stainless steel, other metal, ceramic, woven mesh, silicone, plastics, other polymeric materials, filter paper fibres, hemp fibres, or similar fibres that maintain integrity through the vapour generation phase at a temperature necessary to vaporize the API from the micro sponge. The sachet is prepared from material that will not combust, melt, vaporize, degrade or otherwise result in vaporized material, vaporized or otherwise mobilized breakdown byproducts, or other flowable material at temperatures used to generate vapour. More durable materials for the container may facilitate larger containers with greater volumes of the particulate complex within the container.

The particulate complex is contained within a porous vapour-permeable capsule, other capsule or other rigid container made of a container material such as stainless steel, other metal, ceramic, woven mesh, silicone, plastics, other polymeric materials, filter paper fibres, hemp fibres, or similar fibres or other suitably rigid material for ease of dosing, administration and other handling of the particulate complex. The rigid container may be reinforced where the container material is paper or other more flexible material. The container includes slots, perforations or other apertures for providing fluid communication between a chamber within the container and the external environment. The apertures are of an aperture size smaller than the particle size of the particulate complex, and also smaller than the particle size of the exhausted crosslinked polysaccharide, may be applied to sequester the micro sponge within the container, both while bound with API and once exhausted.

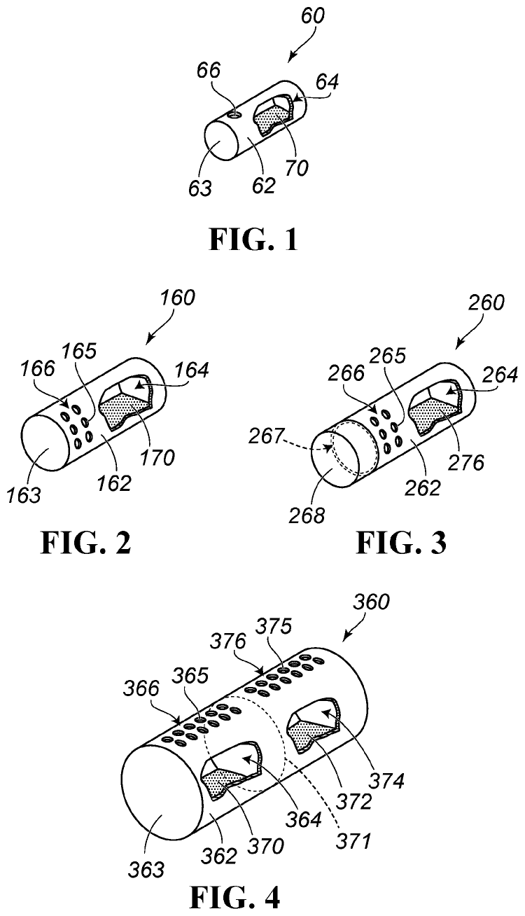

FIG. 1 shows a partial cut-away view of a combination product 60. The combination product 60 includes a container 62 and a particulate complex 70 contained within the container 62. The container 62 includes a container body 63. A chamber 64 is defined within the container body 63. An aperture 66 provides fluid communication between the chamber 64 and an external environment outside the chamber 64. The particulate complex 70 is sequestered inside the container 62 for vaporization, allowing vaporized API from the particulate complex to flow out of the chamber 64 by the aperture 66 for inhalation by a user.

The aperture 66 has a diameter for a circular aperture 66, or a largest dimension for a non-circular aperture 66, that is smaller than the smallest dimension of a particle at the minimum particle size of the particulate complex 70, for restricting flow of the particulate complex 70 through the aperture 66 and facilitating flow of vapour comprising the API from the chamber 64. The container body 63 is formed from a container body material has a vaporization temperature, combustion temperature and melting temperature that are each higher than an API vaporization temperature at which the API vaporizes from the particulate complex 70.

FIG. 2 shows a partial cut-away view of a combination product 160. The combination product 160 includes the particulate complex 170 within the chamber 164. The container 162 includes the container body 163. The chamber 164 is defined within the container body 163. The aperture 166 includes a plurality of individual apertures 165. The aperture 166 provides fluid communication between the chamber 164 and an external environment outside the chamber 164. The container body 163 may be crimped shut during manufacturing of the combination product 160, sequestering the particulate complex 170 inside the container 162 for vaporization, allowing vaporized API from the particulate complex to flow out of the chamber 164 by the aperture 166 for inhalation by a user.

Each individual aperture 165 has a diameter for a circular individual aperture 165, or a largest dimension for a non-circular individual aperture 165, that is smaller than the smallest dimension of a particle at the minimum particle size of the particulate complex 170 for restricting flow of the particulate complex 170 through the individual apertures 165 while facilitating flow of vapour comprising the API from the chamber 164. The container body 163 is formed from a container material that has a vaporization temperature, combustion temperature and melting temperature that are each higher than an API vaporization temperature at which the API vaporizes from the particulate complex 170.

FIG. 3 shows a partial cut-away view of a combination product 260 with a sealed lid 268 over a mouth 267 providing fluid communication with the chamber 264, and including a mixed particulate complex 276. The mixed particulate complex 276 includes an additional payload, in addition to the API. In some embodiments, the API and the additional payload are bound with the same crosslinked polysaccharide. The additional payload may include an additional API or an excipient. The excipient may include an adjuvant, an antiadherant, a binder, a coating, a colour, a disintegrant, a glidant, a lubricant, a preservative, a sorbent, or a compound for imparting a flavour or aroma.

In some embodiments, the API is bound with the crosslinked polysaccharide, providing the particulate complex, while the additional payload is bound with an additional crosslinked polysaccharide, providing an additional particulate complex, and the mixed particulate complex 276 includes both the particulate complex and the additional particulate complex. The additional crosslinked polysaccharide has an additional vaporization temperature, additional combustion temperature and additional melting temperature that are each higher than an additional payload vaporization temperature at which the additional payload vaporizes from the additional particulate complex.

The particulate complex and the additional particulate complex may be chemically distinct crosslinked polysaccharide micro sponges or chemically identical crosslinked polysaccharide micro sponges. The crosslinked polysaccharide and the additional crosslinked polysaccharide may be crosslinked with different ratios of crosslinker to cyclic polysaccharide monomeric unit. A crosslinker crosslinking the crosslinked polysaccharide may be chemically distinct from an additional linker crosslinking the additional crosslinked polysaccharide. A cyclic polysaccharide monomeric unit or other polysaccharide in the crosslinked polysaccharide may be chemically distinct from an additional cyclic polysaccharide monomeric unit, or from non-cyclic polysaccharides in the additional crosslinked polysaccharide.

The combination product 260 includes the mixed particulate complex 276 within the chamber 264. The container 262 includes the container body 263. The chamber 264 is defined within the container body 263. The aperture 266 includes the plurality of individual apertures 265. The aperture 266 provides fluid communication between the chamber 264 and an external environment outside the chamber 264. The mixed particulate complex 276 may be added to chamber 264 through the mouth 267, then the lid 268 may be sealed over the mouth 268, during manufacturing of the combination product, sequestering the mixed particulate complex 276 inside the container 262 for vaporization, allowing vaporized API and vaporized additional API from the mixed particulate complex 276 to flow out of the chamber 264 by the aperture 266 for inhalation by a user.

Each individual aperture 265 has a diameter for a circular individual aperture 265, or a largest dimension for a non-circular individual aperture 265, that is smaller than the smallest dimension of a particle at the minimum particle size of the mixed particulate complex 276 for restricting flow of the mixed particulate complex 276 through the individual apertures 265 while facilitating flow of vapour comprising the API, and vapour comprising the additional payload, from the chamber 264. The container body 263 is formed from a container material that has a vaporization temperature, combustion temperature and melting temperature that are each higher than an API vaporization temperature at which the API vaporizes from the mixed particulate complex 276, each higher than an additional vaporization temperature at which the additional payload vaporizes from the mixed particulate complex 276.

Some APIs are incompatible with each other or with excipients present in a formulation. Manufacturing each of two or more incompatible APIs or additional payloads by binding each API or additional payload to a separate crosslinked polysaccharide may facilitate preparing a stabile formulation by physically separating the APIs during binding with micro sponges in order to prevent interactions between the separate APIs. Once bound with the micro sponges, the resulting particulate complex including the API, and the resulting additional particulate complex including the additional payload may be combined in a single formulation as the mixed particulate complex 276. Combining known volumes of the particulate complex including the API, with the additional particulate complex including the additional API, and mixing the particulate complex and the additional particulate complex to known dosages of the API and the additional payload, facilitates simultaneously releasing both the API and the additional payload by vaporization into a single dosage to be inhaled.

FIG. 4 shows a partial cut-away view of combination product 360 with an additional chamber 374. The combination product 360 includes the particulate complex 370 within the chamber 364. The container 362 includes the container body 363. The chamber 364 is defined within the container body 363. The aperture 366 includes the plurality of individual apertures 365. The aperture 366 provides fluid communication between the chamber 364 and an external environment outside the chamber 364. The particulate complex is sequestered inside the container 362 for vaporization, allowing vaporized API from the particulate complex 370 to flow out of the chamber 364 by the aperture 366 for inhalation by a user.

The combination product 360 includes an additional particulate complex 370 within the additional chamber 374. The additional chamber 374 is defined within the container body 363. An additional aperture 376 includes a plurality of individual additional apertures 375. The additional aperture 376 provides fluid communication between the additional chamber 374 and an external environment outside the additional chamber 374. The additional particulate complex 372 is sequestered within inside the additional chamber 374 for vaporization, allowing vaporized API from the particulate complex 372 to flow out of the additional chamber 374 by the additional aperture 376 for inhalation by a user.

Each individual aperture 365 has a diameter for a circular individual aperture 365, or a largest dimension for a non-circular individual aperture 365, that is smaller than the smallest dimension of a particle at the minimum particle size of the particulate complex 370, for restricting flow of the particulate complex 370 through the individual apertures 365 while facilitating flow of vapour comprising the API from the chamber 364. Each additional individual aperture 375 has a diameter for a circular additional individual aperture 375, or a largest dimension for a non-circular individual aperture 375, that is smaller than the smallest dimension of a particle at the minimum particle size of the additional particulate complex 372, for restricting flow of the additional particulate complex 372 through the additional individual apertures 375 while facilitating flow of vapour comprising the API from the additional chamber 374. The container body 363 is formed from a container material that has a vaporization temperature, combustion temperature and melting temperature that are each higher than an additional payload vaporization temperature at which the additional payload vaporizes from the additional particulate complex 370.

The chamber 364 is separated from the additional chamber 374 by a divider 371. The divider 371 is a portion of the container body 363 that extends within the container body between chamber 364 and the additional chamber 374 for separating the chamber 364 from the additional chamber 374. The divider 371 allows the API and the additional payload to be vaporized through the separate flow paths provided by the aperture 366 and the additional aperture 376. In some cases, the user may cover one or the other of the aperture 366 and the additional aperture 376. Covering the additional aperture 376 and leaving only the aperture 366 exposed facilitates vaporization and inhalation of the API only. Covering the aperture 366 and leaving only the additional aperture 376 exposed facilitates vaporization and inhalation of the additional payload only. Covering either one of the aperture 366 and the additional aperture 376 may facilitate choices by a user at the time of use which of the features of the combination product 360 to access in a given consumption event.

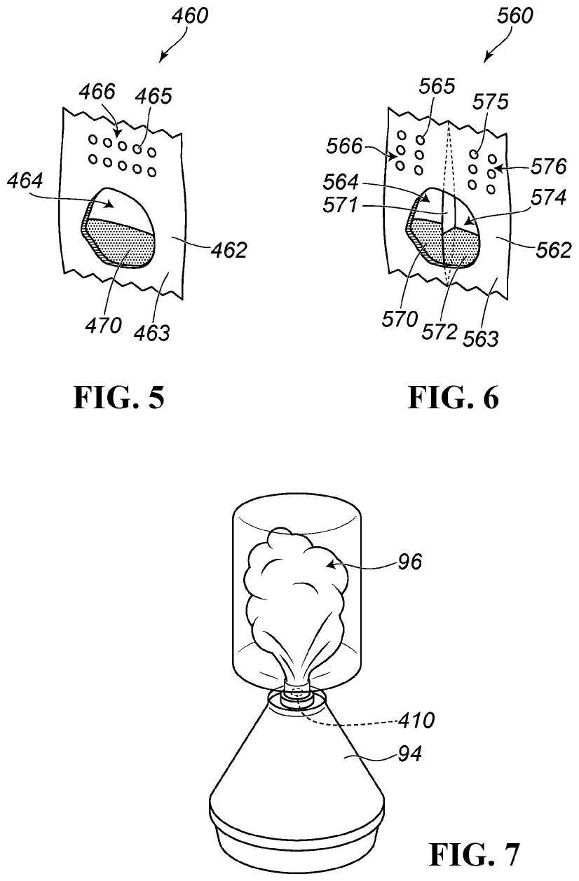

FIG. 5 shows a partial cut-away view of a combination product 460 with a sachet container 462. The combination product 460 includes the particulate complex 470 within the chamber 464. The sachet container 462 includes the sachet container body 463. The chamber 464 is defined within the sachet container body 463. The aperture 466 includes a plurality of individual apertures 465. The aperture 466 provides fluid communication between the chamber 464 and an external environment outside the chamber 464. The sachet container 462 may be sealed during manufacturing of the combination product 460, sequestering the particulate complex inside the sachet container 462 for vaporization, allowing vaporized API from the particulate complex to flow out of the chamber 464 by the aperture 466 for inhalation by a user.

Each individual aperture 465 has a diameter for a circular individual aperture 465, or a largest dimension for a non-circular individual aperture 465, that is smaller than the minimum particle size of the particulate complex 470 for restricting flow of the particulate complex 470 through the individual apertures 465 while facilitating flow of vapour comprising the API from the chamber 464. The sachet container body 463 is formed from a sachet container material that has a vaporization temperature, combustion temperature and melting temperature that are each higher than an API vaporization temperature at which the API vaporizes from the particulate complex 470.

A mixed particulate complex, such as the mixed particulate complex 374 of FIG. 4, may be used in the combination product 460 in place of the particulate complex 470.

FIG. 6 shows a partial cut-away view of a combination product 560 with a sachet container 562 and an additional chamber 574. The combination product 560 includes the particulate complex 570 within the chamber 564. The sachet container 562 includes the sachet container body 563. The chamber 564 is defined within the sachet container body 563. The aperture 566 includes a plurality of individual apertures 565. The aperture 566 provides fluid communication between the chamber 564 and an external environment outside the chamber 564. The particulate complex 570 is sequestered inside the sachet container 562 for vaporization, allowing vaporized API from the particulate complex to flow out of the chamber 564 by the aperture 566 for inhalation by a user.

The combination product 560 includes an additional particulate complex 570 within the additional chamber 574. The additional chamber 574 is defined within the sachet container body 563. The additional aperture 576 includes a plurality of individual additional apertures 575. The additional aperture 576 provides fluid communication between the additional chamber 574 and an external environment outside the additional chamber 574. The additional particulate complex 572 is sequestered within inside the additional chamber 574 for vaporization, allowing vaporized API from the particulate complex 572 to flow out of the additional chamber 574 by the additional aperture 576 for inhalation by a user.

Each individual aperture 565 has a diameter for a circular individual aperture 565, or a largest dimension for a non-circular individual aperture 565, that is smaller than the smallest dimension of a particle at the minimum particle size of the particulate complex 570, for restricting flow of the particulate complex 570 through the individual apertures 565 while facilitating flow of vapour comprising the API from the chamber 564. Each additional individual aperture 575 has a diameter for a circular individual aperture 575, or a largest dimension for a non-circular individual aperture 575, that is smaller than the smallest dimension of a particle at the minimum particle size of the additional particulate complex 572, for restricting flow of the additional particulate complex 572 through the additional individual apertures 575 while facilitating flow of vapour comprising the API from the additional chamber 574. The sachet container body 563 is formed from a container material that has a vaporization temperature, combustion temperature and melting temperature that are each higher than an additional payload vaporization temperature at which the additional payload vaporizes from the additional particulate complex 570.

FIG. 7 shows use of the combination product 460 in a herbal vaporizer 94. Upon heating of the combination product 10 using the herbal vaporizer 94, vapour including the API 96 evolves for inhalation by a user. The herbal vaporizer 94 shown in FIG. 7 uses a bag to receive the vaporized API. The bag may be removed from the herbal vaporizer 94 for inhalation of the vapour including the API 96.

When the combination product 460 is heated, the particulate complex 470 (FIG. 5) is also heated, vaporizing the API. The vapourized API is released to form the particulate complex and passes through the individual apertures 465 (FIG. 5) making up the aperture 466 (FIG. 5) in the vapour-permeable container 460 (FIG. 5) of the combination product 460. During draw, particles of the particulate complex 470 and particles of exhausted crosslinked polysaccharide are retained within the chamber 464 of the container 460 due to having a particle size with a smallest dimension that is larger than the greatest dimension of the individual apertures 465. Upon completion of vapour generation, removal of the dry powder container 460 containing the exhausted micro sponge from the vaporizer 94 for disposal may follow cooling of the sachet body 462. Once the container 460 is cooled, the vaporizer 94 sample chamber may be opened, the exhausted container 460 containing only depleted micro sponge may be discarded, and an unused replacement combination product 460 with the particulate complex 470 may be added to the vaporizer 94 for the next consumption and inhalation event by the user.

Dry vaporization of API from the particulate complex results in a thermal vapour generated without a liquid carrier fluid. Energy introduction by heating using an element on a vaporizer or other inhalation device allows heating of the particulate complex within an air-permeable matrix to generate a thermal vapour of the API that dissociates from the micro sponge. The API may be cooled prior to inhalation by the user. Cooling may take place an inert passageway linking the heating coil to an inhalation mouthpiece. Other examples may include liquid-cooled inhalation flow paths.

The inhalation mouthpiece on the vaporizer, or on a bag into which the vaporizer feeds, functions an aerosol exit, providing fluid communication between the cooling element and the user's respiratory system via air flow initiated through inhalation by the user, and which in some cases may be facilitated by the vaporizer's power. A typical dosage of vapourized API may be administered as a single sachet or other container. A dosage may be single inhalation or as a series of inhalations of the API as the API vaporizes and evolves from the micro sponge and passes through the apertures in the sachet or other container. Where the API is administered as a series of inhalations, a consistent or varied amount of vapour may be delivered in each inhalation. The dosage amount of the API as vapour form is generally controlled by the amount of complex in the sachet, with an intended use of that the entire contents of a sachet are consumed in a single consumption event.

API that was previously bound with the crosslinked polysaccharide may be heated with a pre-defined amount of input energy. Upon vaporization and not combustion, thermal vapour of the API may be released from the particulate complex, leaving the crosslinked polysaccharide behind. The vapourized API may enter an air stream created during inhalation for administration. The vapour may have reduced levels of, and in some cases may be free from, byproducts of combustion and excess heating of the API, of the micro sponge or of the container, with limited compounds beyond the API in the resulting inhalation stream. The airflow is heated to a known temperature to enable a known amount of vapour release by the vaporizer hardware. Production of vapour by heating a plant extract or other heterogenous API may maintain much of the pharmacologic synergy resulting from multiple individual compounds of the plant extract in combination. This property of cannabis specifically is known in peer-reviewed literature as the “the entourage effect”.

Using a vapour dose inhaler, dose control may be based on the weight of the particulate complex, and the corresponding weight of the API, to be vapourized. The amount of API can be assessed as a percentage of the weight of the particulate complex, allowing the amount of API inhaled as a vapour to be defined based on the weight of the particulate complex that is vapourized. In some embodiments, CBD, THC, broad spectrum cannabis extract or other cannabis preparations are bound as API with the crosslinked polysaccharide in an amount if up to about 40%. In some embodiments, loading of CBD, THC, other phytocannabinoids, broad spectrum cannabis extract or other cannabis preparations are bound as API with the crosslinked polysaccharide may be between 1% and 40% of the mass of the crosslinked polysaccharide. In some embodiments, loading of the CBD, THC, other phytocannabinoids, broad spectrum cannabis extract or other cannabis preparations are bound as API with the crosslinked polysaccharide may be between 5% and 25% of the mass of the crosslinked polysaccharide. In some embodiments, loading of the CBD, THC, other phytocannabinoids, broad spectrum cannabis extract or other cannabis preparations are bound as API with the crosslinked polysaccharide may be between 10% and 20% of the mass of the crosslinked polysaccharide. In other cases, a dosage range for API may be determined by the binding capacity of the crosslinked polysaccharide for the API.

Low-dose administration of API, including microdosing of THC, CBD, other phytocannabinoids, DMT, 5-MeO-DMT, other tryptamines, nicotine, nicotine salts or other API can be performed by dilution of a dose of the particulate complex with unreacted crosslinked polysaccharide. Formulations across a range of ratios of API to crosslinked polysaccharide may be prepared, with the weight of the particulate complex vapourized being common to all doses, with a smaller dose of vapour being released and consumed. Inhalation of single compound APIs, or defined mixtures of APIs, may be quantifiable and uniform, reducing inconsistency and safety risk caused by vaporized byproducts.

Crosslinked Polysaccharide

Crosslinked polysaccharides are polymers that include two or more cyclic polysaccharide monomeric subunits, or other polysaccharides, connected in a three-dimensional arrangement by a crosslinker. The cyclic polysaccharide monomeric subunits, or the other polysaccharides, may all be chemically identical or may be mixtures of different monomeric subunits. The crosslinkers may be covalently bound with the cyclic polysaccharide monomeric units through reactions between crosslinking agents and polysaccharide monomers, including with difunctionalised crosslinking agents that react with the hydroxyl groups of polysaccharide monomers, resulting in the crosslinked polysaccharide. The crosslinkers may be covalently bound with the non-cyclic polysaccharides through reactions between crosslinking agents and non-cyclic polysaccharides, including with difunctionalised crosslinking agents that react with the hydroxyl groups of non-cyclic polysaccharides, resulting in the crosslinked polysaccharide.

A given crosslinked polysaccharide may include one chemical compounds as a crosslinker or may include multiple different chemical compounds as crosslinkers.

Monomeric units of crosslinked cyclic polysaccharides each include a ring of six individual sugar residues in α-cyclodextrin, seven individual sugar residues in β-cyclodextrin and eight individual sugar residues in γ-cyclodextrin. Monomeric subunits, whether chemically uniform or including more than one individual type of monomeric subunit, are connected with each other through one or more of a variety of crosslinkers. Non-cyclic crosslinked polysaccharides include a variety of chain lengths of monosaccharides that are crosslinked by crosslinkers.

Monomers of cyclic polysaccharides each include a ring of six individual sugar residues in α-cyclodextrin, seven individual sugar residues in β-cyclodextrin and eight individual sugar residues in γ-cyclodextrin. Non-cyclic polysaccharides include a variety of chain lengths of monosaccharides. Monomers of cyclic polysaccharides, and non-cyclic polysaccharides, whether chemically uniform and identical, or including more than one individual type of monomeric subunit or non-cyclic polysaccharide, may react with one or more crosslinking agents to polymerize into crosslinked cyclic polysaccharides or crosslinked polysaccharides.

The crosslinked polysaccharides may include one type of monomeric subunit of cyclic polysaccharide, such as α-cyclodextrin only, β-cyclodextrin only or γ-cyclodextrin only. The crosslinked polysaccharides may include more than one monomeric subunit of cyclic polysaccharide, such as α-cyclodextrin and β-cyclodextrin, α-cyclodextrin and γ-cyclodextrin, β-cyclodextrin and γ-cyclodextrin, or all three of α-cyclodextrin, β-cyclodextrin and γ-cyclodextrin.

The crosslinked polysaccharides may include one type of non-cyclic polysaccharide, such as maltodextrin only, amylose only or cellulose only. The crosslinked polysaccharides may include more than one non-cyclic polysaccharide, such as maltodextrin and amylose, maltodextrin and cellulose, amylose and cellulose or all three of maltodextrin, amylose or cellulose.

The crosslinked polysaccharides may include both cyclic polysaccharides and non-cyclic polysaccharides in any combination.

The extent of crosslinking affects solubility of the crosslinked polysaccharide. A ratio of 5:1 or greater of crosslinker to cyclic polysaccharide monomeric unit may reduce solubility of crosslinked cyclodextrin polymers or other crosslinked polysaccharides in both hydrophilic solvents and hydrophobic solvents. A crosslinked cyclodextrin polymer or other crosslinked polysaccharide prepared with 4:1 or lower ratio equivalents of crosslinker may have greater solubility in both hydrophilic and hydrophobic solvents while maintaining function of the polymer as a scaffold for the API to bind with to provide the particulate complex be vaporized.

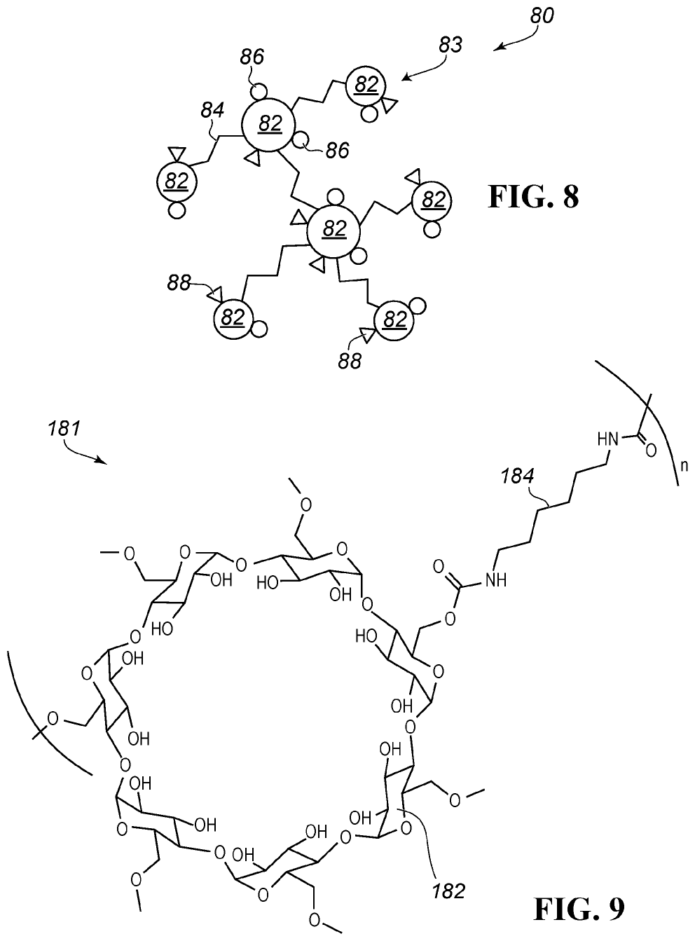

FIG. 8 shows a general schematic of a particulate complex 80 including a crosslinked polysaccharide 83 bound with an API 86 and an additional payload 88. The crosslinked polysaccharide 83 includes a plurality of cyclic polysaccharide monomeric units 82 connected by crosslinkers 84. The API 86 and the additional payload 88 are adsorbed onto, absorbed into, adhered with or otherwise non-covalently bound with the crosslinked polysaccharide 83 at the cyclic polysaccharide monomeric units 82.

The crosslinked polysaccharide 83 has a vaporization temperature, combustion temperature and melting temperature that are each higher than an API vaporization temperature at which the API 86 vaporizes from the additional particulate complex 80. The vaporization temperature, combustion temperature and melting temperature of the crosslinked polysaccharide 83 are each higher than an additional payload vaporization temperature at which the additional payload 88 vaporizes from the additional particulate complex 80.

The crosslinked polysaccharide 83 may be prepared with a variety of monomeric units 82, such as α-cyclodextrin, β-cyclodextrin, γ-cyclodextrin other cyclodextrins, or from other non-cyclic polysaccharides such as amylose, maltodextrin, cellulose, or modified versions of any of the crosslinked polysaccharides. Modified crosslinked polysaccharides may include monomeric units of cyclic polysaccharides or non-cyclic polysaccharides sugars that are alkylated, acetylated, carboxylated, aminated or otherwise modified. Any suitable crosslinked polysaccharide polymer that provides a structure amenable to binding with the API may be applied to preparation of the particulate complex.

The polymeric micro sponge provides a crosslinked polysaccharide 83 binding substrate for the API 86 in the particulate complex 80. The crosslinked polysaccharide 83 is a polymer of polysaccharide monomeric units 82 to provide microparticles that along with the API 86 and the additional payload 88, make up the particulate complex 80. The crosslinked polysaccharide may include a polymer of functionalized polysaccharide monomeric units to provide functionalized microparticle.

The crosslinked polysaccharide 83 may include a cyclodextrin polymer, such as a polymer of α-cyclodextrin monomeric units, β-cyclodextrin monomeric units or γ-cyclodextrin monomeric units. The crosslinked polysaccharide 83 may include a modified cyclodextrin polymer, such as an alkylated cyclodextrin polymer, an acetylated cyclodextrin polymer, a carboxylated cyclodextrin polymer, an aminated cyclodextrin polymer or any suitable crosslinked cyclodextrin polymer. The crosslinked polysaccharide 83 may include a modified α-cyclodextrin polymer, such as an alkylated α-cyclodextrin polymer, an acetylated α-cyclodextrin polymer, a carboxylated α-cyclodextrin polymer, an aminated α-cyclodextrin polymer or any suitable crosslinked α-cyclodextrin polymer. The crosslinked polysaccharide 83 may include a modified β-cyclodextrin polymer, such as an alkylated β-cyclodextrin polymer, an acetylated β-cyclodextrin polymer, a carboxylated β-cyclodextrin polymer, an aminated β-cyclodextrin polymer or any suitable crosslinked β-cyclodextrin polymer. The crosslinked polysaccharide 83 may include a modified γ-cyclodextrin polymer, such as an alkylated γ-cyclodextrin polymer, an acetylated γ-cyclodextrin polymer, a carboxylated γ-cyclodextrin polymer, an aminated γ-cyclodextrin polymer or any suitable crosslinked γ-cyclodextrin polymer.

The crosslinked polysaccharide 83 may include a polymer of amylose, maltodextrin or cellulose. The crosslinked polysaccharide may include a modified polymer of amylose, such as an alkylated amylose polymer, an acetylated amylose polymer, a carboxylated amylose polymer, an aminated amylose polymer or any suitable crosslinked amylose polymer. The crosslinked polysaccharide may include a modified polymer of amylose, such as an alkylated maltodextrin polymer, an acetylated maltodextrin polymer, a carboxylated maltodextrin polymer, an aminated maltodextrin polymer or any suitable crosslinked maltodextrin polymer. The crosslinked polysaccharide may include a modified polymer of amylose, such as an alkylated cellulose polymer, an acetylated cellulose polymer, a carboxylated cellulose polymer, an aminated cellulose polymer or any suitable crosslinked cellulose polymer.

Suitable crosslinking agents include acyl halides, alkyl halides, pseudohalides, esters, diisocyanates, isocyanides and acid anhydrides. The crosslinking agents, and het resulting crosslinkers may provide with different chain lengths. Specific examples of suitable crosslinking agents include hexamethylene diisocyanate (“HMDI”), tetramethylene diisocyanate (“TMDI”), isophorone diisocyanate, 4,4′-methylenebis(phenyl isocyanate), tolylene-2,4-diisocyanate, 1,8-dibromooctane, dimethyl terephthalate, octane-1,8-diol ditosylate, sebacoyl chloride, adipoyl chloride, terephthaloyl chloride, other acyl chlorides, pyromellitic dianhydride, citric acid and epichlorohydrin. The crosslinking agents react with cyclic polysaccharide monomers or with non-cyclic polysaccharides, resulting in a crosslinked polysaccharide with varying degrees of cross linking. The crosslinking agents may be reacted with the cyclic polysaccharide monomers in various ratios of crosslinking agent to cyclic polysaccharide monomer, such as 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. Each of these ratios may be applied to synthesis of any of the crosslinked cyclic polysaccharides disclosed herein.

The crosslinkers resulting from reaction of the crosslinking agents may include hexamethylene dicarbamate, tetramethylene dicarbamate, isophorone dicarbamate, 4,4′-methylenebis(phenyl dicarbamate), tolylene-2,4-dicarbamate, octamethylene, sebacate, adipate, terephthalate, pyromellitate, citrate and 2-hydroxyprop-1,3-yl. The resulting crosslinked polysaccharides may be crosslinked in various ratios of crosslinker to monomeric units of the cyclic polysaccharide in the crosslinked polysaccharide, such as 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1. Each of these ratios may be applied to any of the crosslinked cyclic polysaccharides disclosed herein. Crosslinked cyclodextrins with fewer that four crosslinkers per cyclodextrin may be partially soluble or soluble in water. Cyclodextrins crosslinked with epichlorohydrin may be partially soluble or soluble in water relative to crosslinked cyclodextrins prepared with other crosslinking agents.

The crosslinked polysaccharide may include monomeric subunits of cyclodextrin, alkylated cyclodextrin, acetylated cyclodextrin, carboxylated cyclodextrin, aminated cyclodextrin or any suitable crosslinked cyclodextrin. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with hexamethylene dicarbamate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with tetramethylene dicarbamate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with isophorone dicarbamate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with 4,4′-methylenebis(phenyl dicarbamate). In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with tolylene-2,4-dicarbamate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with octamethylene. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with esters. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with sebacate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with adipate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with terephthalate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with pyromellitate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with citrate. In some embodiments, the monomeric subunits of cyclodextrin, alkylated cyclodextrin, acetylated cyclodextrin, carboxylated cyclodextrin, aminated cyclodextrin or any suitable crosslinked cyclodextrin are crosslinked with 2-hydroxyprop-1,3-yl. The crosslinked polysaccharides may be crosslinked in various ratios of crosslinker to monomeric units of cyclodextrin in the crosslinked cyclodextrin, such as 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1.

The crosslinked polysaccharide may include monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with hexamethylene dicarbamate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with tetramethylene dicarbamate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with isophorone dicarbamate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with 4,4′-methylenebis(phenyl dicarbamate). In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with tolylene-2,4-dicarbamate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with octamethylene. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with esters. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with sebacate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with adipate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with terephthalate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with pyromellitate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with citrate. In some embodiments, the monomeric subunits of α-cyclodextrin, alkylated α-cyclodextrin, acetylated α-cyclodextrin, carboxylated α-cyclodextrin, aminated α-cyclodextrin or any suitable crosslinked α-cyclodextrin are crosslinked with 2-hydroxyprop-1,3-yl. The crosslinked polysaccharides may be crosslinked in various ratios of crosslinker to monomeric units of α-cyclodextrin in the crosslinked α-cyclodextrin, such as 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1.

The crosslinked polysaccharide may include monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with hexamethylene dicarbamate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with tetramethylene dicarbamate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with isophorone dicarbamate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with 4,4′-methylenebis(phenyl dicarbamate). In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with tolylene-2,4-dicarbamate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with octamethylene. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with esters. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with sebacate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with adipate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with terephthalate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with pyromellitate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with citrate. In some embodiments, the monomeric subunits of β-cyclodextrin, alkylated β-cyclodextrin, acetylated β-cyclodextrin, carboxylated β-cyclodextrin, aminated β-cyclodextrin or any suitable crosslinked β-cyclodextrin are crosslinked with 2-hydroxyprop-1,3-yl. The crosslinked polysaccharides may be crosslinked in various ratios of crosslinker to monomeric units of β-cyclodextrin in the crosslinked β-cyclodextrin, such as 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1.

The crosslinked polysaccharide may include monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with hexamethylene dicarbamate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with tetramethylene dicarbamate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with isophorone dicarbamate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with 4,4′-methylenebis(phenyl dicarbamate). In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with tolylene-2,4-dicarbamate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with octamethylene. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with esters. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with sebacate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with adipate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with terephthalate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with pyromellitate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with citrate. In some embodiments, the monomeric subunits of γ-cyclodextrin, alkylated γ-cyclodextrin, acetylated γ-cyclodextrin, carboxylated γ-cyclodextrin, aminated γ-cyclodextrin or any suitable crosslinked γ-cyclodextrin are crosslinked with 2-hydroxyprop-1,3-yl. The crosslinked polysaccharides may be crosslinked in various ratios of crosslinker to monomeric units of γ-cyclodextrin in the crosslinked γ-cyclodextrin, such as 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1.

The crosslinked polysaccharide may include maltodextrin, alkylated maltodextrin, acetylated maltodextrin, carboxylated maltodextrin, aminated maltodextrin or any suitable crosslinked maltodextrin. In some embodiments, the maltodextrin, alkylated maltodextrin, acetylated maltodextrin, carboxylated maltodextrin, aminated maltodextrin or any suitable crosslinked maltodextrin is crosslinked with hexamethylene dicarbamate. In some embodiments, the maltodextrin, alkylated maltodextrin, acetylated maltodextrin, carboxylated maltodextrin, aminated maltodextrin or any suitable crosslinked maltodextrin is crosslinked with tetramethylene dicarbamate. In some embodiments, the maltodextrin, alkylated maltodextrin, acetylated maltodextrin, carboxylated maltodextrin, aminated maltodextrin or any suitable crosslinked maltodextrin is crosslinked with isophorone dicarbamate. In some embodiments, the maltodextrin, alkylated maltodextrin, acetylated maltodextrin, carboxylated maltodextrin, aminated maltodextrin or any suitable crosslinked maltodextrin is crosslinked with 4,4′-methylenebis(phenyl dicarbamate). In some embodiments, the maltodextrin, alkylated maltodextrin, acetylated maltodextrin, carboxylated maltodextrin, aminated maltodextrin or any suitable crosslinked maltodextrin is crosslinked with tolylene-2,4-dicarbamate. In some embodiments, the maltodextrin, alkylated maltodextrin, acetylated maltodextrin, carboxylated maltodextrin, aminated maltodextrin or any suitable crosslinked maltodextrin is crosslinked with octamethylene. In some embodiments, the maltodextrin, alkylated maltodextrin, acetylated maltodextrin, carboxylated maltodextrin, aminated maltodextrin or any suitable crosslinked maltodextrin is crosslinked with esters. In some embodiments, the maltodextrin, alkylated maltodextrin, acetylated maltodextrin, carboxylated maltodextrin, aminated maltodextrin or any suitable crosslinked maltodextrin is crosslinked with sebacate. In some embodiments, the maltodextrin, alkylated maltodextrin, acetylated maltodextrin, carboxylated maltodextrin, aminated maltodextrin or any suitable crosslinked maltodextrin is crosslinked with adipate. In some embodiments, the maltodextrin, alkylated maltodextrin, acetylated maltodextrin, carboxylated maltodextrin, aminated maltodextrin or any suitable crosslinked maltodextrin is crosslinked with terephthalate. In some embodiments, the maltodextrin, alkylated maltodextrin, acetylated maltodextrin, carboxylated maltodextrin, aminated maltodextrin or any suitable crosslinked maltodextrin is crosslinked with pyromellitate. In some embodiments, the maltodextrin, alkylated maltodextrin, acetylated maltodextrin, carboxylated maltodextrin, aminated maltodextrin or any suitable crosslinked maltodextrin is crosslinked with citrate. In some embodiments, the maltodextrin, alkylated maltodextrin, acetylated maltodextrin, carboxylated maltodextrin, aminated maltodextrin or any suitable crosslinked maltodextrin is crosslinked with 2-hydroxyprop-1,3-yl.

The crosslinked polysaccharide may include amylose, alkylated amylose, acetylated amylose, carboxylated amylose, aminated amylose or any suitable crosslinked amylose. In some embodiments, the amylose, alkylated amylose, acetylated amylose, carboxylated amylose, aminated amylose or any suitable crosslinked amylose is crosslinked with hexamethylene dicarbamate. In some embodiments, the amylose, alkylated amylose, acetylated amylose, carboxylated amylose, aminated amylose or any suitable crosslinked amylose is crosslinked with tetramethylene dicarbamate. In some embodiments, the amylose, alkylated amylose, acetylated amylose, carboxylated amylose, aminated amylose or any suitable crosslinked amylose is crosslinked with isophorone dicarbamate. In some embodiments, the amylose, alkylated amylose, acetylated amylose, carboxylated amylose, aminated amylose or any suitable crosslinked amylose is crosslinked with 4,4′-methylenebis(phenyl dicarbamate). In some embodiments, the amylose, alkylated amylose, acetylated amylose, carboxylated amylose, aminated amylose or any suitable crosslinked amylose is crosslinked with tolylene-2,4-dicarbamate. In some embodiments, the amylose, alkylated amylose, acetylated amylose, carboxylated amylose, aminated amylose or any suitable crosslinked amylose is crosslinked with octamethylene. In some embodiments, the amylose, alkylated amylose, acetylated amylose, carboxylated amylose, aminated amylose or any suitable crosslinked amylose is crosslinked with esters. In some embodiments, the amylose, alkylated amylose, acetylated amylose, carboxylated amylose, aminated amylose or any suitable crosslinked amylose is crosslinked with sebacate. In some embodiments, the amylose, alkylated amylose, acetylated amylose, carboxylated amylose, aminated amylose or any suitable crosslinked amylose is crosslinked with adipate. In some embodiments, the amylose, alkylated amylose, acetylated amylose, carboxylated amylose, aminated amylose or any suitable crosslinked amylose is crosslinked with terephthalate. In some embodiments, the amylose, alkylated amylose, acetylated amylose, carboxylated amylose, aminated amylose or any suitable crosslinked amylose is crosslinked with pyromellitate. In some embodiments, the amylose, alkylated amylose, acetylated amylose, carboxylated amylose, aminated amylose or any suitable crosslinked amylose is crosslinked with citrate. In some embodiments, the amylose, alkylated amylose, acetylated amylose, carboxylated amylose, aminated amylose or any suitable crosslinked amylose is crosslinked with 2-hydroxyprop-1,3-yl.

The crosslinked polysaccharide may include cellulose, alkylated cellulose, acetylated cellulose, carboxylated cellulose, aminated cellulose or any suitable crosslinked cellulose. In some embodiments, the cellulose, alkylated cellulose, acetylated cellulose, carboxylated cellulose, aminated cellulose or any suitable crosslinked cellulose is crosslinked with hexamethylene dicarbamate. In some embodiments, the cellulose, alkylated cellulose, acetylated cellulose, carboxylated cellulose, aminated cellulose or any suitable crosslinked cellulose is crosslinked with tetramethylene dicarbamate. In some embodiments, the cellulose, alkylated cellulose, acetylated cellulose, carboxylated cellulose, aminated cellulose or any suitable crosslinked cellulose is crosslinked with isophorone dicarbamate. In some embodiments, the cellulose, alkylated cellulose, acetylated cellulose, carboxylated cellulose, aminated cellulose or any suitable crosslinked cellulose is crosslinked with 4,4′-methylenebis(phenyl dicarbamate). In some embodiments, the cellulose, alkylated cellulose, acetylated cellulose, carboxylated cellulose, aminated cellulose or any suitable crosslinked cellulose is crosslinked with tolylene-2,4-dicarbamate. In some embodiments, the cellulose, alkylated cellulose, acetylated cellulose, carboxylated cellulose, aminated cellulose or any suitable crosslinked cellulose is crosslinked with octamethylene. In some embodiments, the cellulose, alkylated cellulose, acetylated cellulose, carboxylated cellulose, aminated cellulose or any suitable crosslinked cellulose is crosslinked with esters. In some embodiments, the cellulose, alkylated cellulose, acetylated cellulose, carboxylated cellulose, aminated cellulose or any suitable crosslinked cellulose is crosslinked with sebacate. In some embodiments, the cellulose, alkylated cellulose, acetylated cellulose, carboxylated cellulose, aminated cellulose or any suitable crosslinked cellulose is crosslinked with adipate. In some embodiments, the cellulose, alkylated cellulose, acetylated cellulose, carboxylated cellulose, aminated cellulose or any suitable crosslinked cellulose is crosslinked with terephthalate. In some embodiments, the cellulose, alkylated cellulose, acetylated cellulose, carboxylated cellulose, aminated cellulose or any suitable crosslinked cellulose is crosslinked with pyromellitate. In some embodiments, the cellulose, alkylated cellulose, acetylated cellulose, carboxylated cellulose, aminated cellulose or any suitable crosslinked cellulose is crosslinked with citrate. In some embodiments, the cellulose, alkylated cellulose, acetylated cellulose, carboxylated cellulose, aminated cellulose or any suitable crosslinked cellulose is crosslinked with 2-hydroxyprop-1,3-yl.

FIG. 9 shows a β-cyclodextrin monomeric subunit 181 that may be within a crosslinked polysaccharide 183 of β-cyclodextrin monomeric subunit 182 connected with the hexamethylene dicarbamate crosslinkers 184. A single β-cyclodextrin monomeric unit 181 with a single crosslinker 184 is shown. The torus of the β-cyclodextrin monomeric cone is hydrophobic due to the presence of the skeletal carbons and ethereal oxygens that line the torus.

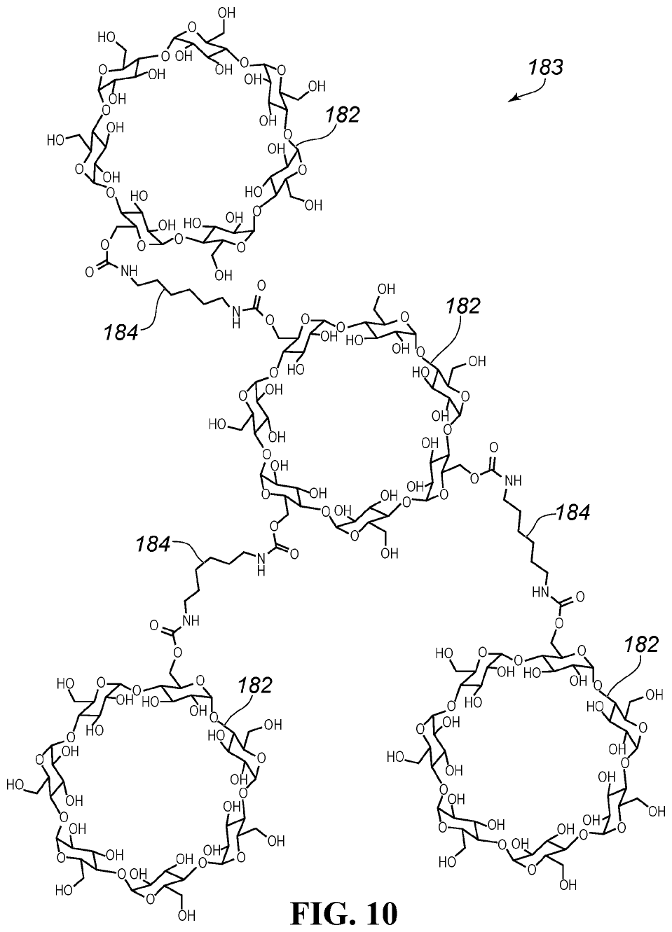

A β-cyclodextrin crosslinked polysaccharide, such as the β-cyclodextrin crosslinked polysaccharide 183 shown in FIG. 10, may include two or more β-cyclodextrin monomeric units 182 crosslinked with each other using hexamethylene dicarbamate as the crosslinkers. The ratio of hexamethylene dicarbamate crosslinkers 184 to β-cyclodextrin crosslinked polysaccharide is at least 1:1. The β-cyclodextrin micro sponge crosslinked polysaccharide may be prepared using seven equivalents or eight equivalents of HMDI per β-cyclodextrin monomer. This results in a with a ratio of hexamethylene dicarbamate crosslinkers 184 to β-cyclodextrin monomeric unit 182 of 7:1 or 8:1.

FIG. 10 shows a β-cyclodextrin crosslinked polysaccharide 183 of the β-cyclodextrin monomeric units 182 with hexamethylene dicarbamate crosslinkers 184, providing a scaffold for the API and the additional payload in the particulate complex, including as shown for the particulate complex 80 in FIG. 8. hexamethylene dicarbamate crosslinkers 184 between multiple β-cyclodextrin monomeric units 182 with multiple hexamethylene dicarbamate crosslinkers 184 are shown in the β-cyclodextrin scaffold crosslinked polysaccharide 183. The β-cyclodextrin scaffold crosslinked polysaccharide 183 reversibly binds with hydrophobic APIs such as phytocannabinoids or with hydrophilic APIs such as nicotine to form the particulate complex.

Without intending to be bound by any theory, the result of the architecture on the β-cyclodextrin crosslinked polysaccharide may include a lipoidal microenvironment within the torus. Binding between APIs and β-cyclodextrin s may be within the tori or outside the tori of the β-cyclodextrin monomeric units. Nicotine, which is hydrophilic, may bind within the torus or with the structure of the crosslinked polysaccharide other than the torus. Hydrophobic compounds may also solubilize either within the torus or with the structure of the crosslinked polysaccharide other than the torus. With β-cyclodextrin monomeric units 182, the lipophilic cavity defined by the interior of the torus may be approximately 262 Angstroms in diameter.

An inclusion complex may result where the API is bound within a torus of a cyclic polysaccharide monomeric subunit. In the case of a crosslinked polysaccharide that is a cyclic polysaccharide assembled from crosslinked β-cyclodextrins, such as α-cyclodextrin, β-cyclodextrin, γ-cyclodextrin or other β-cyclodextrins, binding within a torus of a β-cyclodextrin subunit may be favoured by enthalpy, entropy or both. Without intending to be limited by any theory, where an API has energetically favourable binding as an inclusion complex with a crosslinked β-cyclodextrin, the vaporisation temperature of the API may rise relative to the vaporization temperature of the API when not bound with the crosslinked β-cyclodextrin. As a result, the API may vaporize more slowly from and inclusion complex than the API without the crosslinked β-cyclodextrin.

Binding of an API with the crosslinked polysaccharide may not increase the vaporisation temperature of the API where the API is not bound as an inclusion complex within the torus of the crosslinked polysaccharide. Without intending to be limited by any theory, this may be a result of the API not forming an inclusion complex within the crosslinked polysaccharide monomeric units. Rather, the API may be bound outside the torus of crosslinked polysaccharides within the micro sponge.

As below in Example I, vaporization temperatures for pure CBD that are not bound with crosslinked polysaccharide include an onset of 160° C. When bound with β-cyclodextrin crosslinked polysaccharide that are crosslinked with hexamethylene dicarbamate crosslinkers to provide the particulate complex, CBD vaporized with an onset of 140° C. in Example I.

As below in Example II and III, vaporization temperatures for pure CBG and CBGA that are not bound with crosslinked polysaccharide include an onset of 190° C. for both CBG vaporization and CBG vaporization following decarboxylated from CBGA. When bound with β-cyclodextrin crosslinked polysaccharide that are crosslinked with hexamethylene dicarbamate crosslinkers, CBG vaporized with an onset of 220° C. in both Examples II from pure CBG in the particulate complex, and Example III following decarboxylation from pure CBGA in the particulate complex.

In Example IV, CBD vaporized from a β-cyclodextrin crosslinked polysaccharide crosslinked with sebacate crosslinkers with an onset of 120° C.

In Example V, CBD vaporized from a β-cyclodextrin crosslinked polysaccharide crosslinked with adipate crosslinkers with an onset of 100° C.

In Example VI, CBD vaporized from a β-cyclodextrin crosslinked polysaccharide crosslinked with terephthalate crosslinkers with an onset of 120° C.

Preparation of Particulate Complexes

Porous micro sponges prepared from the crosslinked polysaccharides are polymeric and may often be insoluble, particularly with greater degrees of crosslinking. The micro sponge facilitates binding with API. The API may be present in solution or in liquid form, and may be bound with the crosslinked polysaccharide through contact with the micro sponge. Binding, whether by absorption, adsorption, adhering or other non-covalent binding may be a result of chemical properties, such as hydrophobic interactions driven by entropic forces. The non-covalent binding is a reversible process determined by the surface energy of the material relative to the surrounding environment.

Methods are provided herein for binding API with a crosslinked polysaccharide micro sponge, forming the particulate complex between the API and the micro sponge. External conditions that promote physical state changes to bind the API with the crosslinked polysaccharide may be applied. The API may be bound with at a known concentration on the crosslinked polysaccharide to form the particulate complex between the API and the crosslinked polysaccharide. The complex may be filtered out of an antisolvent solution and washed.

Removing water from the particulate complex may facilitate use of the composition in medical applications. Removing water may also facilitate formulating the resulting composition into solid dosage forms, including dosage forms for vaporization. Once dry, the particulate complex is in the form of a free powder, containing a known quantity of bound API per gram of complex. After binding of the API to the crosslinked polysaccharide and washing away hydrophilic extract components and solvent, the API that was bound by the crosslinked polysaccharide may be freeze dried or otherwise formulated into a stable dry powder formulation.

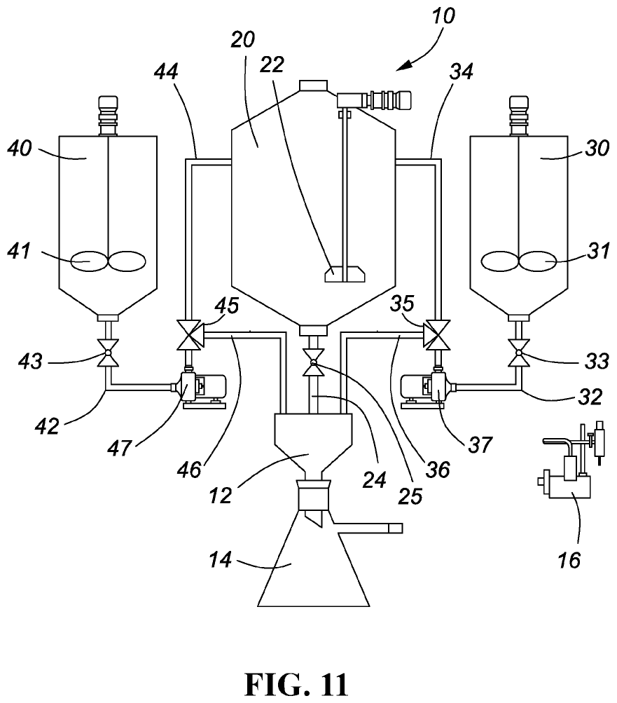

The below methods shown in FIGS. 11 to 18 are disclosed in relation to the API for simplicity. The below methods also apply to the additional payloads, including an additional API or an excipient. Where an additional payload is being added, then the API 54 would be replaced with the additional payload, or the API 54 and the additional payload would be added together where the properties of the API 54 and the additional payload are consistent with use of a single pair of solvent 50 and antisolvent 55, and with a consistent mixing process for the solvent 50 and antisolvent 55.

FIG. 11 shows a binding system 10. The system 10 includes a binding vessel 20. A filter 12 is in fluid communication with the binding vessel 20 for receiving fluid from the binding vessel 20 and filtering material out of the fluid. The filter 12 is shown as a filter funnel but any suitable filter may be applied (e.g. a sintered glass filter, polytetrafluoroethylene membrane filter, etc.) A recovery vessel 14 is in fluid communication with the filter 12 for receiving filtrate that passes through the filter 12. The recovery vessel 14 is shown as a Büchner funnel, but any suitable recovery vessel 14 may be applied (e.g. a flask, Erlenmeyer, round-bottom flask, beaker, test tube, etc.). A processing system 16 may be in fluid communication with the recovery vessel 14 for processing API and micro sponge complex that is captured using the filter 12. The binding vessel 20 is in fluid communication with a solvent vessel 30 for receiving solvent from the solvent vessel 30. The binding vessel 20 is in fluid communication with an antisolvent vessel 40 for receiving antisolvent from the antisolvent vessel 40.

Each of the binding vessel 20, the solvent vessel 30 and the antisolvent vessel 40 may be any suitable fluid vessel appropriate for the size, scale and application of the system 10 (e.g. a tank, pressure-rated tank, etc.).

The solvent 50 may be any suitable solvent in which the API is soluble, and that will not damage the API or the crosslinked polysaccharide. In some cases, the crosslinked polysaccharide may be insoluble in the solvent. For APIs that include phytocannabinoids, the solvent may comprise a lipophilic solvent. Suitable lipophilic solvents may include alcohol (e.g. methanol, ethanol, n-propyl alcohol, isopropyl alcohol, etc.), other polar organic solvents (e.g. acetone, acetonitrile, tetrahydrofuran, glycerol, DMSO, dichloromethane, chloroform, etc.), eutectic solvents (e.g. equimolar mixture of acetic acid and menthol, glucose syrup, etc.), ionic liquids (e.g. 1-butyl-3-methylimidazolium tetrafluoroborate, etc.), supercritical CO₂ and hydrocarbons (e.g. n-hexane, butane, propane, etc.). The lipophilic solvent may include a suitable combination of any of the above solvents.

The antisolvent 55 may be any suitable antisolvent in which the API is insoluble or poorly soluble, and that will not damage the API or the crosslinked polysaccharide. In some cases, the crosslinked polysaccharide may be insoluble in the antisolvent. For APIs that include phytocannabinoids, the antisolvent may comprise a hydrophilic antisolvent. The hydrophilic antisolvent may for example include water, brine, salt solutions or buffered solutions, including solutions comprising a chelating agent.

The solvent and the antisolvent are defined in terms of hydrophobicity and hydrophilicity relative to each other and not necessarily on any particular scale of hydrophobicity and hydrophilicity. For a given lipophilic target compound and a given sample, the solvent and the antisolvent may be selected to be miscible with each other for facilitating recovery of the lipophilic target compound using the crosslinked polysaccharide as described above. Where the solvent and the antisolvent are not miscible with each other to any great degree, the solvent may be evaporated by increasing heat or by decreasing pressure prior to addition of antisolvent instead of being mixed with the antisolvent.

The binding vessel 20 includes an agitator 22 positioned within the binding vessel 20. The agitator 22 is for agitating a fluid inside the binding vessel 20 (e.g. the agitator 22 is shown in FIG. 13 mixing the contact mixture 52). The agitator 22 is shown as a rotary stirring agitator but any suitable agitator may be used (e.g. cross-flow, a venturi, static agitator, etc.). The binding vessel 20 is in fluid communication with the filter 12 through an output flow line 24, and fluid communication between the binding vessel 20 and the output flow line 24 may be engaged and disengaged by an output valve 25.

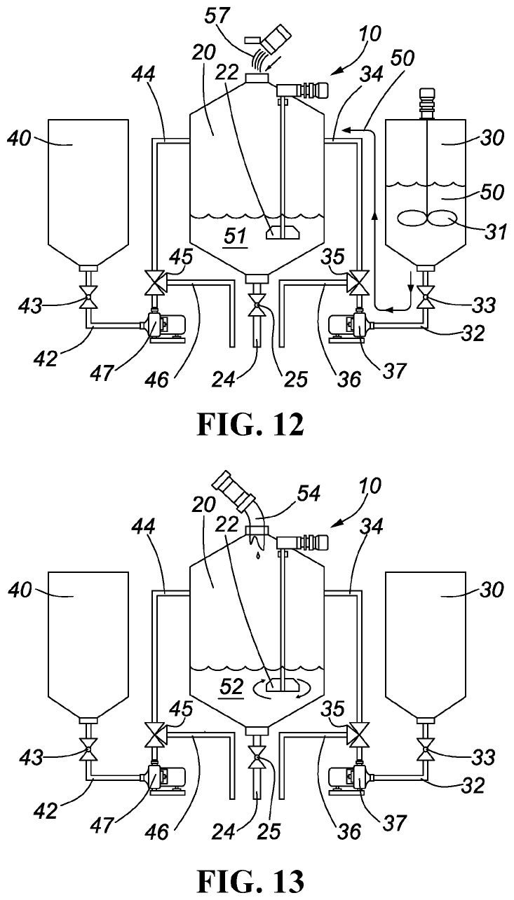

The solvent vessel 30 includes an agitator 31 positioned within the solvent vessel 30. The agitator 31 is for agitating a solvent (e.g. the agitator 31 is shown agitating the solvent 50 in FIG. 12, etc.) inside the solvent vessel 30 to mix the solvent. The solvent vessel 30 is in fluid communication with the binding vessel 20 and with the filter 12.

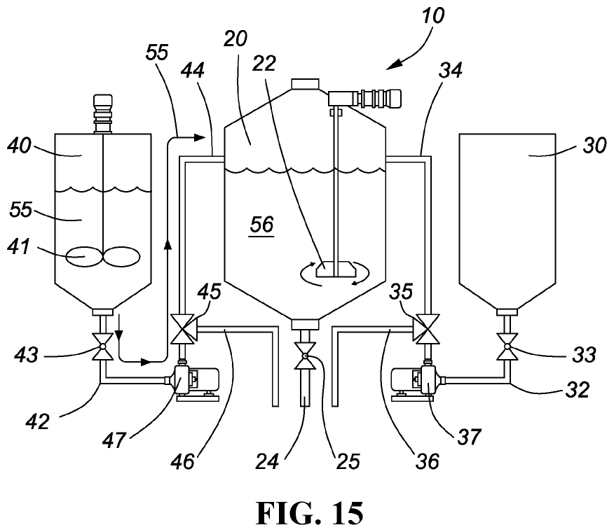

The antisolvent vessel 40 includes an agitator 41 positioned within the antisolvent vessel 40. The agitator 41 is for agitating an antisolvent (e.g. the agitator 41 is shown agitating the antisolvent 55 in FIG. 15, etc.) inside the antisolvent vessel 40 to mix the antisolvent. The antisolvent vessel 40 is in fluid communication with the binding vessel 20 and with the filter 12.

The solvent vessel 30 may be in fluid communication with the binding vessel 20 through an upstream solvent flow line 32 and a downstream solvent flow line 34. Fluid communication between the solvent vessel 30 and the binding vessel 20 may be provided and broken by an upstream solvent valve 33 and a downstream solvent valve 35. Fluid communication between the solvent vessel 30 and the binding vessel 20 may be driven by a pump 37.

The solvent vessel 30 may be in fluid communication with the filter 12 through an upstream solvent flow line 32 and a solvent rinse flow line 36. Fluid communication between the solvent vessel 30 and the filter 12 may be provided and broken by the upstream solvent valve 33 and the downstream solvent valve 35. Fluid communication between the solvent vessel 30 and the filter 12 may be driven by the pump 37.

The antisolvent vessel 40 may be in fluid communication with the binding vessel 20 through an upstream antisolvent flow line 42 and a downstream antisolvent flow line 44. Fluid communication between the antisolvent vessel 40 and the binding vessel 20 may be provided and broken by an upstream antisolvent valve 43 and a downstream antisolvent valve 45. Fluid communication between the antisolvent vessel 40 and the binding vessel 20 may be driven by a pump 47.

The antisolvent vessel 40 may be in fluid communication with the filter 12 through an upstream antisolvent flow line 42 and an antisolvent rinse flow line 46. Fluid communication between the antisolvent vessel 40 and the filter 12 may be provided and broken by the upstream antisolvent valve 43 and the downstream antisolvent valve 45. Fluid communication between the antisolvent vessel 40 and the filter 12 may be driven by the pump 47.

Binding Protocol

FIGS. 12 to 17 show the system 10 in use to load an API 54 on to micro sponge 57 to provide a particulate complex, using a solvent 50 and an antisolvent 55. The solvent 50 is stored in and sourced from the solvent vessel 30. The antisolvent 55 is stored in and sourced from the antisolvent vessel 40. For simplicity of review of FIGS. 12 to 17, the solvent 50 and the agitator 31 are shown in the solvent vessel 30 only when the solvent 50 is being supplied to the binding vessel 20. Similarly, and also for simplicity of review of FIGS. 12 to 17, the antisolvent 55 and the agitator 41 are shown in the antisolvent vessel 40 only when the antisolvent 55 is being supplied to the binding vessel 20. In figures where these solvents are not being supplied to the binding vessel 20, the solvent vessel 30 and the antisolvent vessel 40 are shown without detail.

In FIG. 12, the micro sponge 57 is provided into the binding vessel 20. The micro sponge 57 may be supplied dry, for example as a powder, and the binding vessel 20 may be chilled prior to addition of the micro sponge 57.

The micro sponge 57 is combined with the solvent 50 in the binding vessel 20 to provide a slurry 51. The solvent 50 may be provided to the binding vessel 20 from the solvent vessel 30 via the upstream solvent flow line 32 and the downstream solvent flow line 34. The solvent 50 may be provided in a ratio of 75% micro sponge 57 to 25% solvent 50. Alternatively, either a portion of the micro sponge 57 or all of the micro sponge 57 may be added to the binding vessel 20 after adding the antisolvent 55 to the binding vessel 20. Depending on the adsorbent 73 and the antisolvent 55 that are used, ratios of adsorbent 73:solvent 50 may range from 10:90, 9:91, 8:92, 7:93, 6:94, 5:95, 4:96, 3:97, 2:98 or 1:99.

FIG. 13 shows the API 54 being loaded into the binding vessel 20 and combined with the slurry 51, providing a contact mixture 52. The binding vessel 20 may be chilled to between 3° C. and room temperature, such as 4° C., when the API 54 is added to the binding vessel 20. In some cases, lower temperatures may also facilitate maintaining liquidity of a low boiling gaseous solvent, such as butane or other shorter hydrocarbon solvents with vaporization temperatures below or close to 20° C. In some cases, lower temperatures may also improve the stability of temperature-sensitive lipophilic target compounds. In some cases, higher temperatures may be applied to decrease solvent viscosity. In some cases, higher temperatures may be used to facilitate in situ decarboxylation of phytocannabinoids, if decarboxylated phytocannabinoids are the target molecule and where decarboxylation was not previous carried out on the API 54. Temperature may also be modulated to maintain a temperature range at which supercritical fluids have the appropriate physical properties.

The API 54 includes at least one target compound. The API 54 may include for example an extract or other sample from a biological source (e.g. a plant, fungi, yeast, bacteria, or other microorganism). The target compound may include any compound that complexes with, binds with or otherwise adsorbs to the micro sponge 57. The target compound may adsorb with the micro sponge 57 in some cases by coordinating within a torus formed by the molecular structure of monomeric cyclic polysaccharide units within the crosslinked polysaccharides of the micro sponge 57, or by binding with the micro sponge 57 outside of the torus.

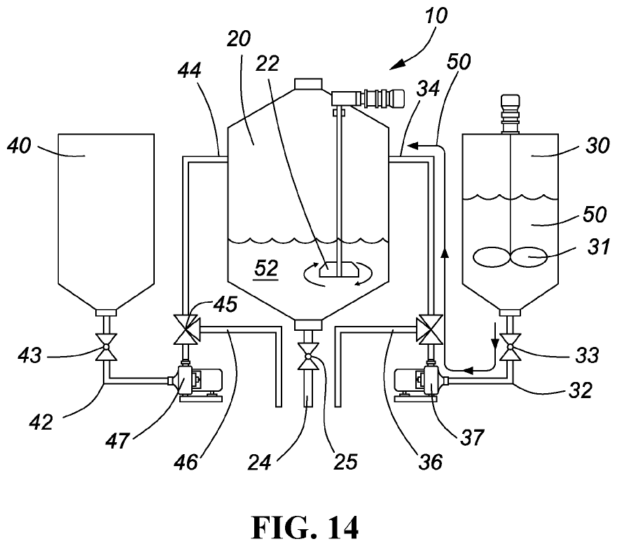

FIG. 14 shows additional solvent 50 being added to the binding vessel 20 from the solvent vessel 30 to combine with the contact mixture 52 via the upstream solvent flow line 32 and the downstream solvent flow line 34. In some cases, the additional solvent 50 may dilute any water or other solvents that may have been included in the API 54. The additional solvent 50 may facilitate dissolution of target compounds that may be present in the API 54. The contact mixture 52 may be agitated by the agitator 22.

FIG. 15 shows the antisolvent 55 being added to the binding vessel 20 from the antisolvent vessel 40. The antisolvent 55 may be added to the binding vessel 20 via the upstream antisolvent flow line 42 and the downstream antisolvent flow line 44 and combined with the contact mixture 52 to provide a binding mixture 56. Where the target compound includes a phytocannabinoid, the API 54 may be an ethanolic extract of Cannabis sativa flowers or other trichrome-bearing biomass (or from any plant commonly associated with cannabis from within the genus Cannabis based on interpretation of taxonomy of the C. sativa plant and its varieties) the solvent 50 may be ethanol and the antisolvent 55 may be water, the binding mixture 56 may target a ratio of 30:70 solvent 50 to antisolvent 55 for driving the lipophilic target compounds into the micro sponge 57 core. Other ratios of solvent 50 to antisolvent 55 for the binding mixture 56 may be selected for other solvents 60, antisolvents 70, samples 54 or target lipophilic compounds. Together, the solvent 50 and the antisolvent 55 in a ratio that pushes the API 54 into the micro sponge 57 provide a binding solvent 58. The binding solvent 58 may include miscible solvent 50 and antisolvent 55 or immiscible solvent 50 and antisolvent 55 separated into two layers. Ratios of solvent 50:antisolvent 55 may range from 95:5, 90:10, 85:15, 80:20, 75:25, 70:30, 65:35, 60:40, 55:45, 50:50, 45:55, 40:60, 35:65, 30:70, 25:75, 20:80, 15:85, 10:90 and 5:95.

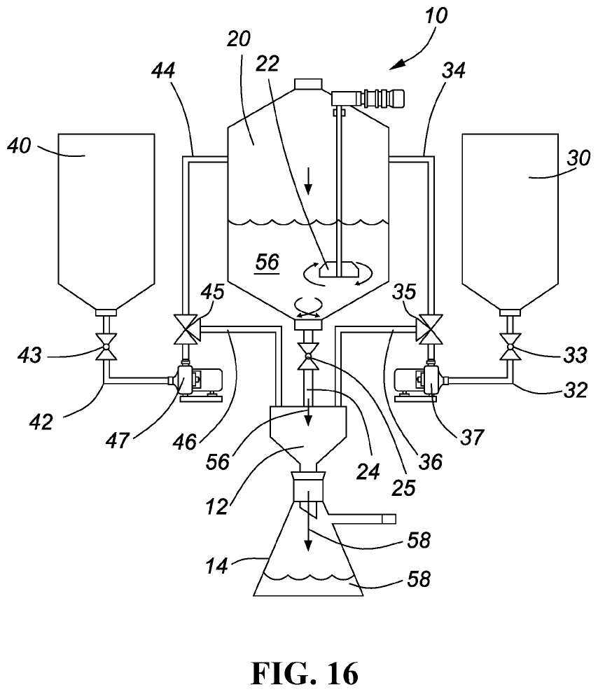

FIG. 16 shows the binding mixture 56 being run through the filter 12 for filtering and retaining the micro sponge 57 with captured lipophilic target compounds. The binding solvent 58 runs through the filter 12 into the recovery vessel 14.

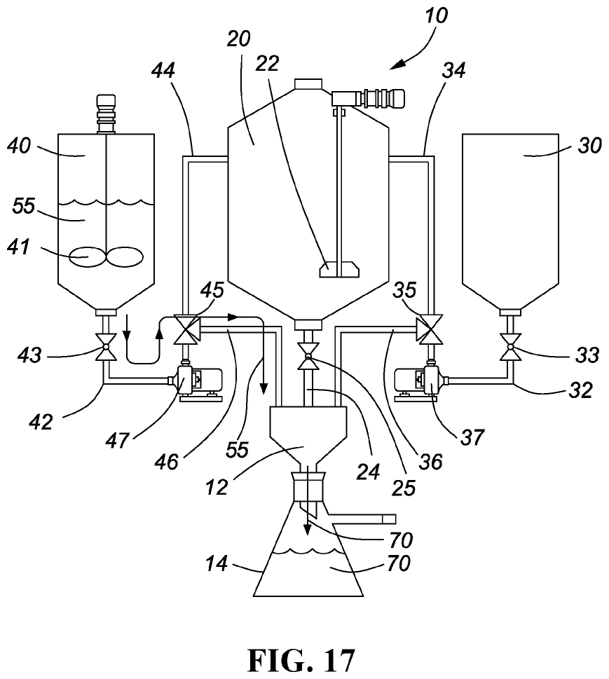

FIG. 17 shows rinsing of the filter 12 with antisolvent 55 to wash the filter 12 via the upstream antisolvent flow line 42 and the antisolvent rinse flow line 46. An amount of antisolvent 55 used to wash the filter 12 may be about 3 or 4 times the volume of the binding mixture 56 that was passed through the filter 12.

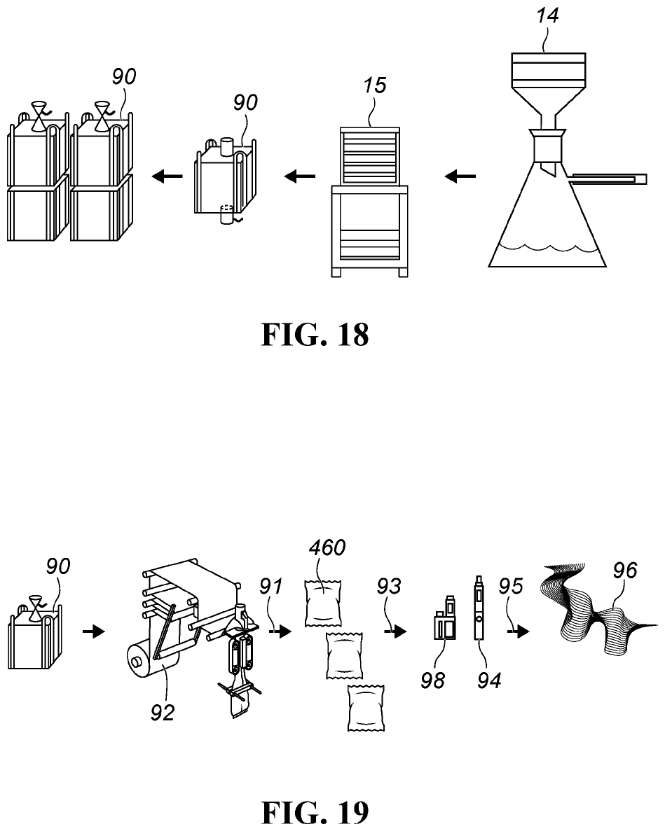

FIG. 18 is a schematic diagram showing storage of a particulate complex provided by the system of FIG. 11 and the method of FIGS. 12 to 17. The particulate complex is recovered from the recovery vessel 14 and placed in a desiccator 15 to remove any residual antisolvent 55. Once the particulate complex is dried, the particulate complex is transferred to a storage vessel 90 for long-term stable storage. The API within the particulate complex may remain stable for longer periods of time than the API alone.

FIG. 19 shows manufacture and use of sachets including the API-micro sponge complex, similarly to the combination product 460. The particulate complex is retrieved from the storage vessel 90. A sachet manufacturing system 92 is used in a manufacturing process 91 to prepare the combination product 460 by filling the particulate complex into the combination products 460 using an automated sachet filling and sealing machine. The combination product 460 is loaded 93 into a vaporization device. In FIG. 19, the herbal vaporization device 94 and a nicotine vaporization device 98 are each shown as examples. Other suitable vaporization devices or other thermal vapour delivery devices may be applied as appropriate for a given combination product. The combination products 460 are heated in the appropriate herbal vaporization device 94, nicotine vaporization device 98 or other vaporization device, to produce the vapour including the API 96.

Method Used in Examples

Crosslinked β-cyclodextrin micro sponges were prepared by crosslinking β-cyclodextrin monomers using HMDI, sebacoyl chloride, adipoyl chloride and terephthaloyl chloride as crosslinking agents.

Insoluble hexamethylene dicarbamate-crosslinked β-cyclodextrin micro sponges were prepared. A mixture of β-cyclodextrin (2.0 g, 1 equivalent) and dibutyltin dilaurate (one drop) in dimethylformamide (“DMF”) (15 mL) was stirred under a nitrogen atmosphere until a clear solution was formed. A solution of HMDI (2.26 mL, 8 equivalents) in DMF (5 mL) was added dropwise to the solution of β-cyclodextrin and dibutyltin dilaurate. The resulting reaction mixture was heated at 70° C. for 24 h under a nitrogen atmosphere, forming a viscous gel. The reaction mixture was cooled to 25° C. and the resulting hexamethylene dicarbamate-crosslinked β-cyclodextrin micro sponge was precipitated by pouring the reaction mixture into 500 mL chloroform. Where necessary, a spatula was used to break up the gel and more chloroform was added to mobilise the remaining gel for transfer. The resulting suspension was stirred for 12 h. The precipitate was collected by filtration and resuspended in 500 mL of deionised water. The precipitated was collected by filtration and dried in an oven at 60° C. over night. The dried hexamethylene dicarbamate-crosslinked β-cyclodextrin micro sponge was ground by ball-milling and sieved to obtain a target particle size, which may for example include between about 63 μm and about 250 μm.

In other experiments, whose data is not shown in Examples I to VI, the β-cyclodextrin was dried in an oven at 170° C. for one hour immediately prior to use, and 1.98 mL for 7:1 equivalent of HMDI crosslinking agent to cyclic polysaccharide were used.

Insoluble acyl-chloride-crosslinked β-cyclodextrin micro sponges were prepared. Acyl chlorides used were sebacoyl chloride, adipoyl chloride and terephthaloyl chloride. β-cyclodextrin was dried in an oven at 170° C. for one hour immediately prior to use. A mixture of β-cyclodextrin (1.98 g, 1 equivalent) in N-Methyl-2-pyrrolidone (“NMP”) (15 mL) was stirred under a nitrogen atmosphere and cooled to 0° C. Acid chloride (7 equivalents) was added in one portion. The resulting reaction mixture was stirred at 0° C. for 30 minutes and then heated to 60° C. for 16 hours, during which time the reaction mixture formed a gel of insoluble acyl-chloride-crosslinked β-cyclodextrin micro sponges in NMP. After cooling the reaction mixture, deionised water (50 mL) was added to the reaction mixture, and the reaction mixture gel was broken up mechanically using a spatula. This mixture was stirred for 30 minutes then filtered and washed with 200 mL of deionised water. Ethanol (100 mL) was added to the solid filtrate. The insoluble acyl-chloride-crosslinked β-cyclodextrin micro sponges were stirred for 3 hours, then filtered and washed with 200 mL of ethanol. Deionised water (100 mL) was added to the solid filtrate. The resulting mixture of insoluble acyl-chloride-crosslinked β-cyclodextrin micro sponge in water was stirred for 16 hours. The insoluble acyl-chloride-crosslinked β-cyclodextrin micro sponges were again collected by filtration and dried in an over at 60° C. overnight. Dried acyl-chloride-crosslinked β-cyclodextrin micro sponge was ground by ball-milling and sieved to obtain the required particle sizes, which may for example include between about 63 μm and about 250 μm.

API-crosslinked micro sponge complex was prepared using a similar method for each of the crosslinked β-cyclodextrin micro sponges, including the hexamethylene dicarbamate-crosslinked β-cyclodextrin micro sponges and the acyl-chloride-crosslinked β-cyclodextrin micro sponges. Detailed steps specific to each example are provided below under the individual Examples I to VI. Generally, one equivalent of the API is dissolved in approximately three times the minimum amount of a solvent. Five to ten equivalents of the crosslinked micro sponge are added to the solution and stirred. An antisolvent for the API is added dropwise to this mixture until the API first begins to precipitate out of solution. The volume of the antisolvent at which the API begins to precipitate defines a “precipitation onset volume” of antisolvent. Antisolvent is added dropwise until a volume of the antisolvent equivalent to a further two precipitation onset volumes of the antisolvent. Once three precipitation onset volumes total of the antisolvent are added, the mixture is stirred until no more precipitated API is visible in the mixture. Then the mixture is filtered to collect the solid API-crosslinked micro sponge complex, washed with the antisolvent, and dried under vacuum.

All phytocannabinoids used as API, including CBG, CBGA and CBD were either sourced commercially or chemically synthesized. Other phytocannabinoids with comparable vaporization points and comparable complexation chemistry to CBG, CBGA and CBD may also be bound with the micro sponges to form the API-crosslinked polysaccharide complex. The other phytocannabinoids include THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA.

Differential scanning calorimetry (“DSC”) and thermogravimetric analysis (“TGA”) was performed on a Netzsch STA 409PC/PG with Infrared (“IR”) analysis of the volatiles under a purge of nitrogen gas. TGA was completed at a temperature of 250° C.

Vaporisation of cannabinoids from an insoluble acyl-chloride-crosslinked β-cyclodextrin micro sponge was performed on a Storz and Bickel Volcano Hybrid set to 200° C.

The suitability of an API for vaping from the insoluble β-cyclodextrin micro sponge, or from other crosslinked polysaccharide micro sponges, can be tested using TGA. When TGA is applied, a sample of API bound with the insoluble β-cyclodextrin micro sponge is heated at a constant rate, simulating the heat used in a vaporisation device. The sample is kept under a constant flow of nitrogen gas, simulating the inhalation of the user, drawing volatile compounds away from the heated sample.

TGA of a sample of API bound with an insoluble β-cyclodextrin micro sponge shows mass loss corresponding to the mass of API bound with the insoluble β-cyclodextrin micro sponge as a function of heating time. Vaporization kinetics were observed in these methods. Vaporization kinetics refer to the rate of dissociation of the API as vapour from the micro sponge, as determined by TGA.

IR spectroscopy analysis of the nitrogen stream can be used to confirm the presence of the volatised API.

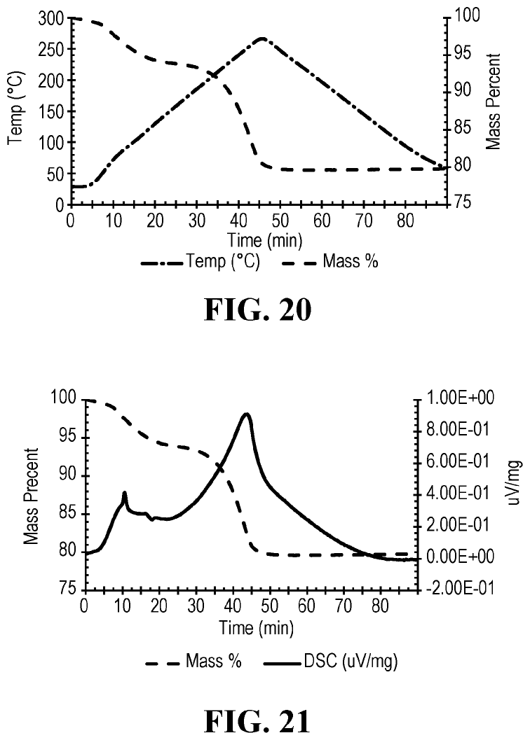

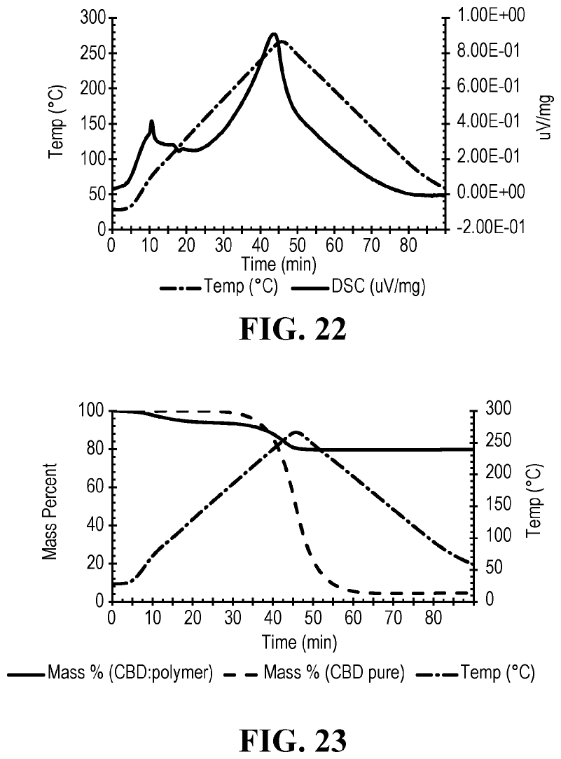

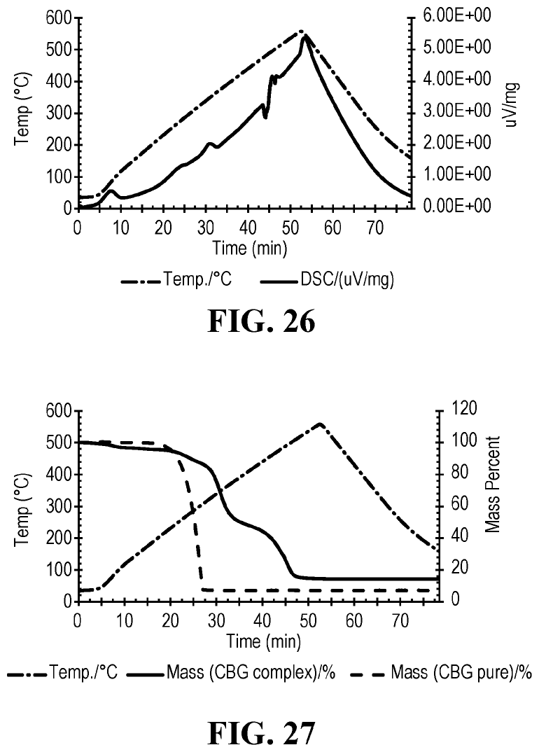

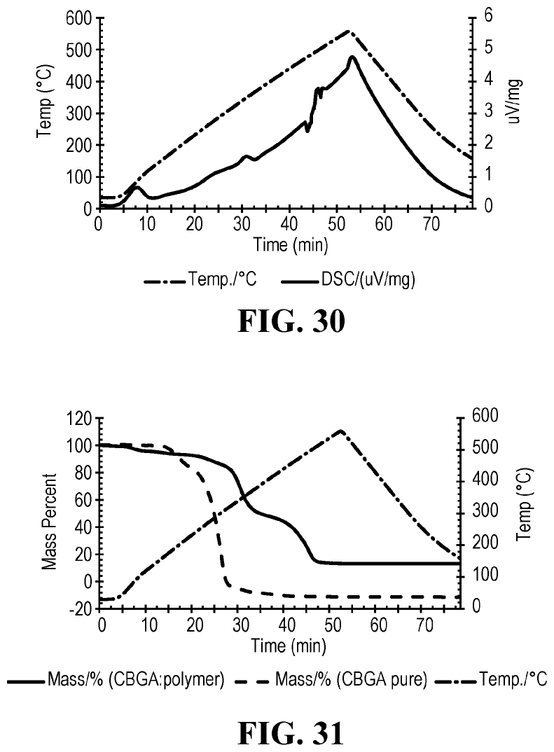

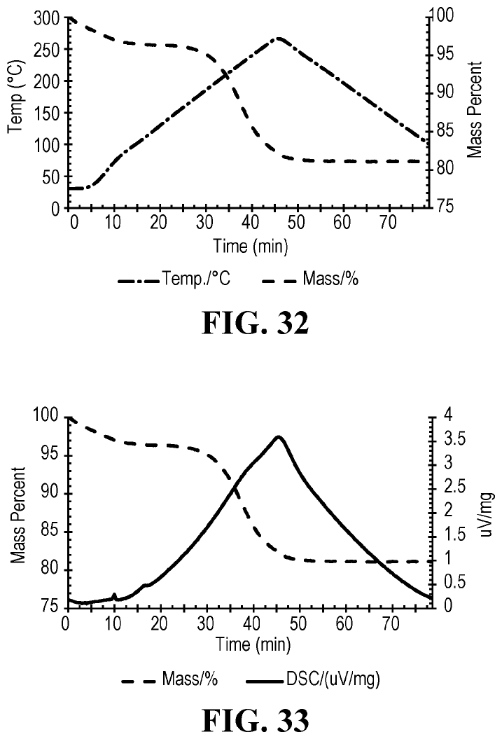

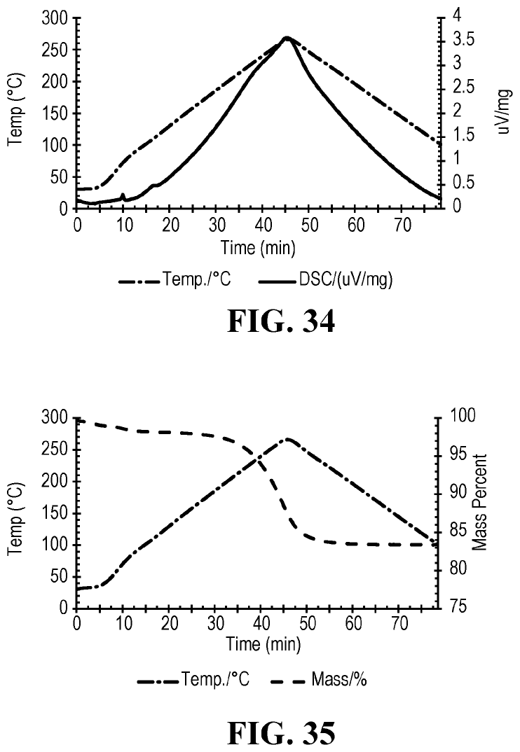

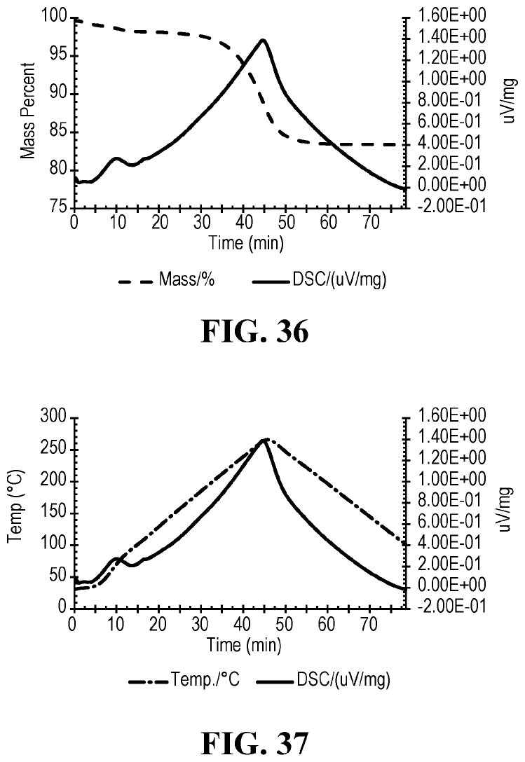

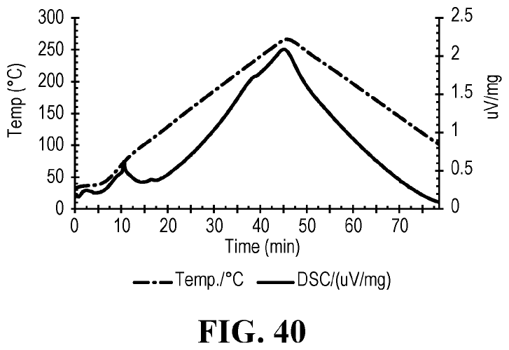

In the presentation of data for each of Examples I, II and III, four graphs are shown with y axes as indicated in each example in FIGS. 20 to 31, as in the below Table 1. In the presentation of data for each of Examples IV, V and VI, three graphs are shown with y axes as indicated in FIGS. 32 to 40, as in the below Table 1. In all cases, the x-axis is time in minutes.

TABLE 1
Data shown in FIGS. 20 to 40

| FIGS. | Primary (Left) Y-Axis | Secondary (Right) Y-Axis |
|---|---|---|
| 20/24/28 | Temperature (dot dash line) | Mass Percent (dashed line) |
| 21/25/29 | Mass Percent (dashed line) | DSC (uV/mg) (solid line) |
| 22/26/30 | Temperature (dot dash line) | DSC (uV/mg) (solid line) |
| 23/27/31 | Mass Percent | Temperature (dot-dash line) |
| | API from Complex (solid line) | |
| | Crystalline API (dashed line) | |
| 32/35/38 | Temperature (dot dash line) | Mass Percent (dashed line) |
| 33/36/39 | Mass Percent (dashed line) | DSC (uV/mg) (solid line) |
| 34/37/40 | Temperature (dot dash line) | DSC (uV/mg) (solid line) |

In previous cases, complexation of CBD with cyclodextrin may result in a higher vaporisation temperature than is observed with pure CBD. A higher vaporization temperature was observed in Lv (2019) in a study on shows cyclodextrin-CBD complexes, where mass was lost at higher temperatures in TGA than CBD alone. In contrast, In Examples I, IV, V and VI below, using crosslinked polysaccharides, the TGA traces of CBD complexed with crosslinked polysaccharide vaporize at a lower temperature compared with vaporization of pure CBD.

Pure CBD in Example I vaporized at 160° C. CBD complexed with hexamethylene dicarbamate-crosslinked cyclodextrin in Example I vaporized at 140° C. CBD complexed with sebacoyl-crosslinked cyclodextrin in Example IV vaporized at 120° C. CBD complexed with adipoyl-crosslinked cyclodextrin in Example V vaporized at 100° C. CBD complexed with terephthalate-crosslinked cyclodextrin in Example VI vaporized at 120° C.

Pure CBG in Example II vaporized at 190° C. CBG complexed with hexamethylene dicarbamate-crosslinked cyclodextrin in Example II vaporized at 220° C. Pure CBGA in Example III vaporized at 190° C. as CBG after decarboxylation to CBG. CBGA complexed with hexamethylene dicarbamate-crosslinked cyclodextrin in Example III vaporized at 220° C. as CBG after decarboxylation to CBG.

The losses in mass are occurring at the same temperatures, in addition to the same points in time, as demonstrated with a variety of crosslinked polysaccharides complexed with CBD (FIGS. 23 and 32 to 40), CBG (FIG. 27) and CBGA (FIG. 31).

The suitability of an API for vaping from the insoluble β-cyclodextrin micro sponge, or from other crosslinked polysaccharide micro sponges, can also be tested by heating in a commercial cannabis vaporiser. In Examples IV to VI, a sample of CBD bound with insoluble acyl-chloride-crosslinked-β-cyclodextrin micro sponges is heated at 200° C. in a Storz and Bickel Volcano for 5 minutes with the fan setting set to “on”. Analysis of the sample before and after heating using HPLC provides data of the amount of CBD vaporized from the sample during heating.

Table 2 shows the percentage of CBD vaporized in Examples IV to VI as measured on a Storz and Bickel Volcano vaporizer at 200° C.

TABLE 2
CBD Vaporized in Examples IV to VI

| Example | Crosslinker | CBD Vaporized |
|---|---|---|
| IV | Sebacoyl chloride | 63% |
| V | Adipoyl chloride | 68% |
| VI | Terephthaloyl chloride | 62% |

### Example 1

Binding of CBD isolate with an insoluble hexamethylene dicarbamate-crosslinked β-cyclodextrin micro sponge and vaporization of CBD from the resulting complex was assessed.

To a solution of 1.4 g of CBD in 53 ml of absolute ethanol in was added 7 g of the insoluble hexamethylene dicarbamate-crosslinked β-cyclodextrin micro sponge with a particle size of 125 to 250 μm.

This mixture was stirred using a magnetic stir bar at 400 rpm, whilst 370 mL of deionised water was added over 75 minutes. After water addition was complete, the mixture was stirred for a further 18 hours.

The CBD/insoluble hexamethylene dicarbamate-crosslinked β-cyclodextrin micro sponge complex was isolated by vacuum filtration of the reaction mixture followed by washing with deionised water. 8.3 g of CBD/insoluble β-cyclodextrin micro sponge complex was recovered. The CBD content of the CBD/insoluble hexamethylene dicarbamate-crosslinked β-cyclodextrin micro sponge complex was analysed by HPLC and found to be 17% by weight.

FIGS. 20 to 22 show TGA at a ramp rate of 10° C. min⁻¹ of a 32 mg sample of CBD/insoluble hexamethylene dicarbamate-crosslinked β-cyclodextrin micro sponge complex. After an initial loss of bound water (6% of sample mass), the sample loses 14% of its mass, corresponding to volatised CBD, with an onset temperature of 140° C. (21 to 45 minutes).

FIG. 23 shows TGA of CBD bound with the hexamethylene dicarbamate-crosslinked β-cyclodextrin micro sponge compared with TGA of pure, crystalline CBD under the same conditions. The traces show vaporisation of pure CBD with an onset of 160° C., and vaporisation of CBD complexed with the hexamethylene dicarbamate-crosslinked crosslinked β-cyclodextrin micro sponge with an onset of 160° C.

### Example II

Binding of CBG isolate with an insoluble hexamethylene dicarbamate-crosslinked β-cyclodextrin micro sponge and vaporization of CBG from the resulting complex was assessed.

To a solution of 50 mg of CBG in 3.75 ml of absolute ethanol in was added 500 mg of the hexamethylene dicarbamate-crosslinked insoluble β-cyclodextrin micro sponge with a particle size of 63 to 125 μm.

This mixture was stirred using a magnetic stir bar at 400 rpm, whilst 11.25 mL of deionised water was added over 9 minutes. After water addition was complete, the mixture was stirred for a further one hour.

The CBG/hexamethylene dicarbamate-crosslinked insoluble β-cyclodextrin micro sponge complex was isolated by vacuum filtration of the reaction mixture followed by washing with deionised water. 500 mg of CBG/hexamethylene dicarbamate-crosslinked insoluble β-cyclodextrin micro sponge complex was recovered. The CBG content of the CBG/hexamethylene dicarbamate-crosslinked insoluble β-cyclodextrin micro sponge complex was analysed by HPLC, and found to be 10% by weight.

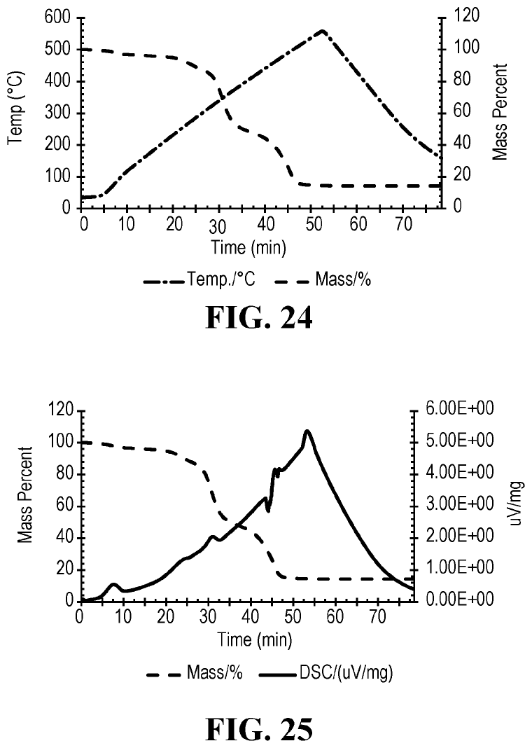

FIGS. 24 to 26 show TGA at a ramp rate of 5° C. min⁻¹ of a 20 mg sample of CBG/hexamethylene dicarbamate-crosslinked insoluble β-cyclodextrin micro sponge complex. After an initial loss of bound water (3% of sample mass), the sample loses 7% of its mass, corresponding to volatised CBG, with an onset temperature of 220° C. (20-26 minutes). The hexamethylene dicarbamate-crosslinked insoluble β-cyclodextrin micro sponge then begins to decompose in two stages, between 26 and 36 minutes (onset temperature 300° C.), and 34 minutes and 48 minutes (onset temperature 400° C.).

FIG. 27 shows TGA of CBG bound with the crosslinked β-cyclodextrin micro sponge compared with TGA of pure, crystalline CBG under the same conditions. The traces show vaporisation of pure CBG with an onset of 190° C., and vaporisation of CBG complexed with the hexamethylene dicarbamate-crosslinked crosslinked β-cyclodextrin micro sponge with an onset of 220° C.

### Example III

Binding of CBGA isolate with an insoluble hexamethylene dicarbamate-crosslinked β-cyclodextrin micro sponge and vaporization of CBGA from the resulting complex was assessed.

To a solution of 50 mg of CBGA in 3.75 ml of absolute ethanol in was added 500 mg of the hexamethylene dicarbamate-crosslinked insoluble β-cyclodextrin micro sponge with a particle size of 63 to 125 μm.

This mixture was stirred using a magnetic stir bar at 400 rpm, whilst 11.25 mL of deionised water was added over 9 minutes. After water addition was complete, the mixture was stirred for a further one hour.

The CBGA/hexamethylene dicarbamate-crosslinked insoluble β-cyclodextrin micro sponge complex was isolated by vacuum filtration of the reaction mixture followed by washing with deionised water. 517 g of CBGA/hexamethylene dicarbamate-crosslinked insoluble β-cyclodextrin micro sponge complex was recovered. The CBGA content of the CBGA/hexamethylene dicarbamate-crosslinked insoluble β-cyclodextrin micro sponge complex was analysed by HPLC and found to be 9.5% by weight.

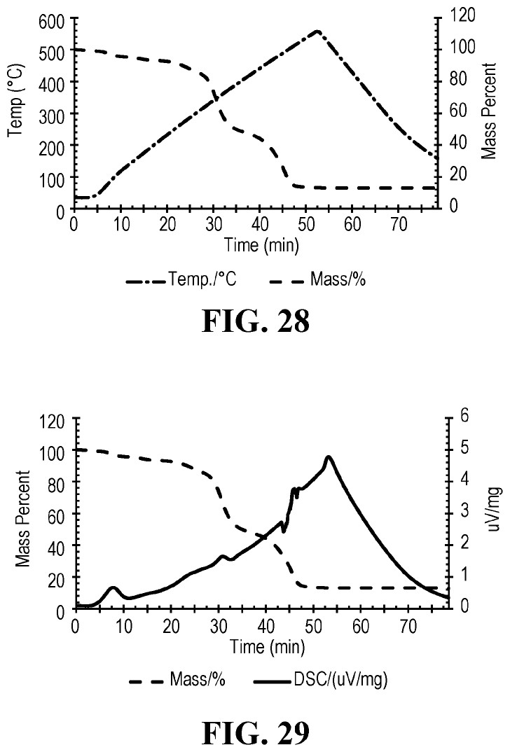

FIGS. 28 to 30 show TGA at a ramp rate of 5° C. min⁻¹ of a sample of 26.8 mg of CBGA/hexamethylene dicarbamate-crosslinked insoluble β-cyclodextrin micro sponge complex. After an initial loss of bound water (4% of sample mass), followed by decarboxylation, the sample loses 7% of its mass, corresponding to volatised CBG, with an onset temperature of 220° C. (18-26 minutes). The hexamethylene dicarbamate-crosslinked insoluble β-cyclodextrin micro sponge then begins to decompose in two stages, between 26 and 36 minutes (onset temperature 300° C.), and 34 minutes and 48 minutes (onset temperature 400° C.).

FIG. 31 shows TGA of CBGA bound with the crosslinked β-cyclodextrin micro sponge compared with TGA of pure, crystalline CBGA under the same conditions. The traces show vaporisation of CBG from pure CBGA with an onset of 190° C., and vaporisation of CBG from CBGA complexed with the hexamethylene dicarbamate-crosslinked crosslinked β-cyclodextrin micro sponge with an onset of 220° C.

### Example IV

Binding of CBD isolate with an insoluble sebacoyl-crosslinked β-cyclodextrin micro sponge and vaporization of CBD from the resulting complex was assessed.

To a solution of 200 mg of CBD in 7.5 ml of absolute ethanol in was added 1.00 g of the sebacoyl-crosslinked insoluble β-cyclodextrin micro sponge with a particle size of 63 to 250 μm.

This mixture was stirred using a magnetic stir bar at 400 rpm, whilst 22.5 mL of deionised water was added over nine minutes. After water addition was complete, the mixture was stirred for a further one hour.

The CBD/sebacoyl-crosslinked insoluble β-cyclodextrin micro sponge complex was isolated by vacuum filtration of the reaction mixture followed by washing with deionised water and drying under vacuum. 1.15 g of CBD/sebacoyl-crosslinked insoluble β-cyclodextrin micro sponge complex was recovered. The CBD content of the CBD/sebacoyl-crosslinked insoluble β-cyclodextrin micro sponge complex was analysed by HPLC and found to be 16% by weight.

FIGS. 32 to 34 show TGA at a ramp rate of 5° C. min⁻¹ of a sample of 7.6 mg of CBD/sebacoyl-crosslinked insoluble β-cyclodextrin micro sponge complex. After an initial loss of bound water (3.6% of sample mass), the sample loses 15% of its mass, corresponding to volatised CBD, with an onset temperature of 120° C. (18 to 50 minutes).

The above process was applied to 100 mg of CBD, resulting in 1.01 g of CBD/sebacoyl-crosslinked insoluble β-cyclodextrin micro sponge complex that was analysed by HPLC and found to be 9% by weight.

100 mg of CBD/sebacoyl-crosslinked insoluble β-cyclodextrin micro sponge complex was placed in a Storz & Bickel dosing capsule and heated at 200° C. for 5 minutes with the fan set to ‘on’. After cooling, the CBD content of the CBD/sebacoyl-crosslinked insoluble β-cyclodextrin micro sponge complex was analysed by HPLC it was found that 63% of the CBD had been vapourized.

### Example V

Binding of CBD isolate with an insoluble adipoyl-crosslinked β-cyclodextrin micro sponge and vaporization of CBD from the resulting complex was assessed.

To a solution of 200 mg of CBD in 7.5 ml of absolute ethanol in was added 1.00 g of the adipoyl-crosslinked insoluble β-cyclodextrin micro sponge with a particle size of 63 to 250 μm.

This mixture was stirred using a magnetic stir bar at 400 rpm, whilst 22.5 mL of deionised water was added over nine minutes. After water addition was complete, the mixture was stirred for a further one hour.

The CBD/adipoyl-crosslinked insoluble β-cyclodextrin micro sponge complex was isolated by vacuum filtration of the reaction mixture followed by washing with deionised water and drying under vacuum. 1.20 g of CBD/adipoyl-crosslinked insoluble β-cyclodextrin micro sponge complex was recovered. The CBD content of the CBD/adipoyl-crosslinked insoluble β-cyclodextrin micro sponge complex was analysed by HPLC and found to be 15% by weight.

FIGS. 35 to 37 show TGA at a ramp rate of 5° C. min⁻¹ of a sample of 22.7 mg of CBD/adipoyl-crosslinked insoluble β-cyclodextrin micro sponge complex. After an initial loss of bound water (1.6% of sample mass), the sample loses 14.7% of its mass, corresponding to volatised CBD, with an onset temperature of 100° C. (15 to 58 minutes).

The above process was applied to 100 mg of CBD, resulting in 1.02 g of CBD/adipoyl-crosslinked insoluble β-cyclodextrin micro sponge complex that was analysed by HPLC and found to be 9% by weight.

100 mg of CBD/adipoyl-crosslinked insoluble β-cyclodextrin micro sponge complex was placed in a Storz & Bickel dosing capsule and heated at 200° C. for 5 minutes with the fan set to ‘on’. After cooling, the CBD content of the CBD/adipoyl-crosslinked insoluble β-cyclodextrin micro sponge complex was analysed by HPLC it was found that 68% of the CBD had been vapourized.

### Example VI

Binding of CBD isolate with an insoluble terephthaloyl-crosslinked β-cyclodextrin micro sponge and vaporization of CBD from the resulting complex was assessed.

To a solution of 200 mg of CBD in 7.5 ml of absolute ethanol in was added 1.00 g of the terephthaloyl-crosslinked insoluble β-cyclodextrin micro sponge with a particle size of 63-250 μm.

This mixture was stirred using a magnetic stir bar at 400 rpm, whilst 22.5 mL of deionised water was added over nine minutes. After water addition was complete, the mixture was stirred for a further one hour.

The CBD/terephthaloyl-crosslinked insoluble β-cyclodextrin micro sponge complex was isolated by vacuum filtration of the reaction mixture followed by washing with deionised water and drying under vacuum. 1.12 g of CBD/terephthaloyl-crosslinked insoluble β-cyclodextrin micro sponge complex was recovered. The CBD content of the CBD/terephthaloyl-crosslinked insoluble β-cyclodextrin micro sponge complex was analysed by HPLC and found to be 18% by weight.

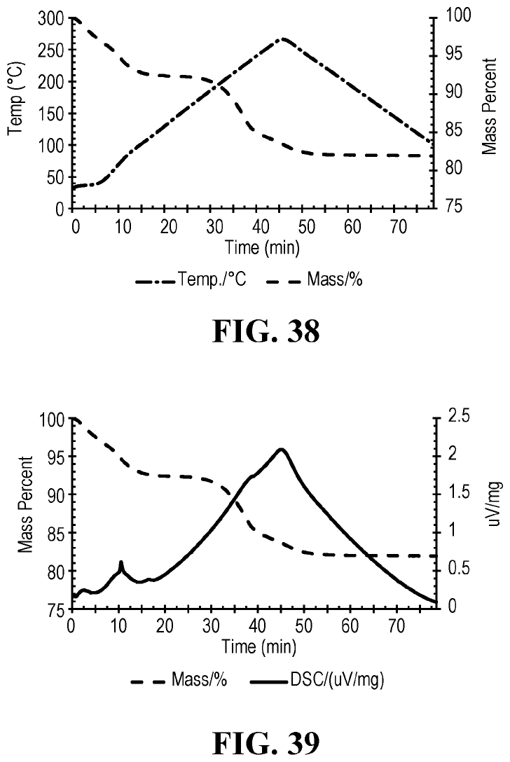

FIGS. 38 to 40 show TGA at a ramp rate of 5° C. min⁻¹ of a sample of 13.5 mg of CBD/terephthaloyl-crosslinked insoluble β-cyclodextrin micro sponge complex. After an initial loss of bound water (7.5% of sample mass), the sample loses 17.9% of its mass, corresponding to volatised CBD, with an onset temperature of 120° C. (18 to 39 minutes).

The above process was applied to 100 mg of CBD, resulting in 1.05 g of CBD/terephthaloyl-crosslinked insoluble β-cyclodextrin micro sponge complex that was analysed by HPLC and found to be 9% by weight.

100 mg of CBD/terephthaloyl-crosslinked insoluble β-cyclodextrin micro sponge complex was placed in a Storz & Bickel dosing capsule and heated at 200° C. for 5 minutes with the fan set to ‘on’. After cooling, the CBD content of the CBD/terephthaloyl-crosslinked insoluble β-cyclodextrin micro sponge complex was analysed by HPLC it was found that 62% of the CBD had been vapourized.

### REFERENCES

Arias M J, Mayano J R, Munoz P, Gines J M, Justo A, Giordano F. Study of Omeprazole-g-cyclodextrin complexation in the solid state Drug. Dev Ind Pharm. (2000) 26: 253-59.
Arima H, Miyaji T, Irie T, Hirayama F, Uekama K. Enhancing effect of hydroxypropyl b-cyclodextrin on cutaneous penetration and activation of Ethyl-4-biphenyl acetate in hairless mouse skin. Eur J Pharm Sci. (2001), 6:53-59
J. Heyder, J. Gebhart, G. Rudolf, C. F. Schiller, W. Stahlhofen. Deposition of particles in the human respiratory tract in the size range 0.005-15 μm. J Aerosol Sci (1986) 17: 811-825.
Joachim Heyder. Deposition of Inhaled Particles in the Human Respiratory Tract and Consequences for Regional Targeting in Respiratory Drug Delivery. Proc Am Thorac Soc (2004) 1:315-320
Tara M. Lovestead** and Thomas J. Bruno. Determination of Cannabinoid Vapor Pressures to Aid in Vapor Phase Detection of Intoxication. Forensic Chem (2017) 5: 79-85.
Hamed Mirzaei, Allen O'Brien, Nishat Tasnim, Adithya Ravishankara, Hamed Tahmooressi, Mina Hoorfar. Topical review on monitoring tetrahydrocannabinol in breath. J. Breath Res. (2020) 14:034002
Pin Lv, Dongjing Zhang, Mengbi Guo, Jing Liu, Xuan Chen, Rong Guo, Yanping Xu, Qingying Zhang, Ying Liu, Hongyan Guo, Ming Yang, Structural analysis and cytotxicity of host-guest inclusion complexes of cannabidiol with three native cyclodextrins. Journal of Drug Delivery Science and Technology, (2019), accepted manuscript copy.
Arima H, Miyaji T, Irie T, Hirayama F, Uekama K. Enhancing effect of hydroxypropyl b-cyclodextrin on cutaneous penetration and activation of Ethyl-4-biphenyl acetate in hairless mouse skin. Eur J Pharm Sci. (2001) 6:53-9
Asai K, Morishita M, Katsuta H, Hosoda S, Shinomiya K, Noro M, Tsuneji N, and Takayama K. The effects of water-soluble cyclodextrins on the histological integrity of the rat nasal mucosa. Int J Pharm. (2002) 246:25-35
Yeongkwon Son, Daniel P Giovenco, Cristine Delnevo, Andrey Khlystov, Vera Samburova, and Qingyu Meng. Indoor Air Quality and Passive E-cigarette Aerosol Exposures in Vape-Shops. Nicotine Tob Res (2020) 22(10): 1772-1779.
Soha Talih, Rola Salman, Rachel El-Hage, Ebrahim Karam, Nareg Karaoghlanian, Ahmad El-Hellani, Najat Saliba, Alan Shihadeh. Characteristics and toxicant emissions of JUUL electronic cigarettes. Tob Control (2019) 28(6):678-680
Shigehisa Uchiyama, Mayumi Noguchi, Ayana Sato, Miho Ishitsuka, Yohei Inaba, and Naoki Kunugita. Determination of Thermal Decomposition Products Generated from E-Cigarettes. Chem. Res. Toxicol (2020), 33(2): 576-583.
Yamasaki, H., Makihata, Y. & Fukunaga, K. Efficient phenol removal of wastewater from phenolic resin plants using crosslinked cyclodextrin particles. J. Chem. Technol. Biotechnol. (2006) 81: 1271-1276

### EXAMPLES ONLY

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A combination product comprising:
a container comprising a container body, a chamber defined within the container body for receiving particulate material, and an aperture defined on the container body for providing fluid communication between the chamber and an external environment; and
a particulate complex received within the chamber, the particulate complex comprising an API bound with a crosslinked polysaccharide, and the particulate complex having a minimum particle size;
wherein a largest dimension of the aperture is smaller than a smallest dimension of a particle having the minimum particle size for restricting flow of the particulate complex through the aperture and facilitating flow of vapour comprising the API from the chamber through the aperture;
the crosslinked polysaccharide has a vaporization temperature, combustion temperature and melting temperature that are each higher than an API vaporization temperature at which the API vaporizes from the particulate complex;
the API comprises CBD;
the crosslinked polysaccharide comprises a β-cyclodextrin cyclic polysaccharide crosslinked with crosslinkers;
the crosslinkers are selected from the group consisting of sebacate, adipate and terephthalate; and
the API vaporization temperature is below 140° C.

2. The combination product of claim 1 wherein the container body comprises a rigid capsule.

3-9. (canceled)

10. The combination product of claim 1 wherein the minimum particle size is 63 μm to 125 μm.

11. The combination product of claim 1 wherein the particulate complex in the chamber provides a single dose of the API.

12. The combination product of claim 1 wherein the API comprises a purified compound.

13. The combination product a claim 1 wherein the API comprises a heterogenous mixture.

14. The combination product of claim 13 wherein the heterogenous mixture comprises a botanical extract, a fungal extract or any other extract from biological material.

15. The combination product of claim 14 wherein the heterogeneous mixture comprises a botanical extract from any plant within the genus Cannabis.

16. The combination product of claim 1 wherein the API comprises a compound selected from the group consisting of a terpenoid, a flavonoid and an alkaloid.

17-19. (canceled)

20. The combination product of claim 1 wherein the API comprises a phytocannabinoid selected from the group consisting of THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA.

21-24. (canceled)

25. The combination product of claim 1, wherein:
the crosslinked polysaccharide comprises a plurality of cyclodextrin monomeric units crosslinked by a cyclodextrin crosslinker;
the crosslinked polysaccharide was produced by reacting cyclodextrin monomers with a cyclodextrin crosslinking agent;
reacting the cyclodextrin monomers with the cyclodextrin crosslinking agent comprises reaction of a ratio of cyclodextrin crosslinking agent to cyclodextrin monomers; and
the ratio of cyclodextrin crosslinking agent to cyclodextrin monomers is selected from the group consisting of 1:1, 2:1, 3:2, 3:1, 4:3, 4:1, 5:4, 5:3, 5:2, 5:1, 6:5, 6:1, 7:6, 7:5, 7:4, 7:3, 7:2, 7:1, 8:7, 8:5, 8:3, 8:1, 9:8, 9:7, 9:5, 9:4, 9:2, 9:1, 10:9, 10:7, 10:3 and 10:1.

26-40. (canceled)

41. The combination product of claim 1 wherein the vaporization temperature, combustion temperature and melting temperature of the crosslinked polysaccharide are each at least 20° C. above the API vaporization temperature.

42. The combination product of claim 1 further comprising an additional payload bound with the crosslinked polysaccharide.

43. The combination product of claim 1, further comprising an additional particulate complex received within the container, the additional particulate complex comprising an additional payload bound with an additional crosslinked polysaccharide, and the additional particulate complex having particles of an additional minimum particle size; and
wherein the additional crosslinked polysaccharide has an additional vaporization temperature, additional combustion temperature and additional melting temperature that are each higher than an additional payload vaporization temperature at which the additional payload vaporizes from the additional particulate complex.

44-51. (canceled)

52. The combination product of claim 43 wherein contact between the API and the additional payload results in a reduced stability of the API or a reduced stability of the additional payload.

53-54. (canceled)

55. The combination product of claim 43 wherein the additional payload comprises an additional API.

56. The combination product of claim 55 wherein the additional API comprises an additional purified compound.

57. The combination product claim 55 wherein the additional API comprises an additional heterogenous mixture.

58. The combination product of claim 57 wherein the additional heterogenous mixture comprises a botanical extract, a fungal extract or any other extract from biological material.

59. The combination product of claim 58 wherein the additional heterogeneous mixture comprises a botanical extract from any plant within the genus Cannabis.

60. The combination product of claim 55 wherein the additional API comprises an additional compound selected from the group consisting of a terpenoid, a flavonoid and an alkaloid.

61. The combination product of claim 55 wherein the additional API comprises an additional compound selected from the group consisting of DMT, 5-MeO-DMT, other tryptamines, nicotine, an amphetamine, ephedrine, pseudoephedrine, other alkaloids, menthol or salts of any of the foregoing.

62. The combination product of claim 55 wherein the additional API comprises an additional hydrophobic compound having an octanol:water partition coefficient of greater than 2.

63. The combination product of claim 62 wherein the additional hydrophobic compound comprises a phytocannabinoid.

64. The combination product of claim 63 wherein the additional API comprises a phytocannabinoid selected from the group consisting of THC, THCA, CBD, CBDA, CBN, CBG, CBGA, CBC, CBCA, CBE, CBEA, CBL, CBLA, iso-THC, iso-THCA, CBT, CBTA, Δ8THC, Δ8THCA, THCV, THCVA, CBDV and CBDVA.

65. The combination product of claim 55 wherein the additional API comprises an additional hydrophilic compound having an octanol:water partition coefficient of 2 or lower.

66. The combination product of claim 43 wherein the additional payload comprises an excipient.

67-125. (canceled)

126. The combination product of claim 1 wherein:
the crosslinked polysaccharide comprises a β-cyclodextrin cyclic polysaccharide crosslinked with sebacate crosslinkers; and
the API vaporization temperature is about 100° C.

127. The combination product of claim 1 wherein:
the crosslinked polysaccharide comprises a β-cyclodextrin cyclic polysaccharide crosslinked with adipate crosslinkers; and
the API vaporization temperature is about 100° C.

128. The combination product of claim 1 wherein:
the crosslinked polysaccharide comprises a β-cyclodextrin cyclic polysaccharide crosslinked with terephthalate crosslinkers; and
the API vaporization temperature is about 120° C.
